(19) United States
(12) Patent Application Publication
(10) Pub. No.: US 2003/0073832 A1
(43) Pub. Date: Apr. 17, 2003

(54) Novel aminophenyl ketone derivatives

(76) Inventor: Sophie Elisabeth Havez, Kobenhavn (DK)

Correspondence Address: BIRCH STEWART KOLASCH & BIRCH, PO BOX 747, FALLS CHURCH, VA, 22040-0747, US

(21) Appl. No.: 10/118,942

(22) Filed: Apr. 10, 2002

Related U.S. Application Data
(60) Provisional application No. 60/282,494, filed on Apr. 10, 2001.

(51) Int. Cl. C07D 279/12; C07D 265/30; C07D 211/06; C07D 241/04

(52) U.S. Cl. 544/59; 544/176; 544/386; 546/226; 548/530

(57) ABSTRACT

Novel heteroaryl aminophenyl ketone derivatives which are inhibitors of MAP kinases, in particular the p38 MAP kinase, are useful as anti-inflammatory agents in the prophylaxis or treatment of inflammatory diseases or conditions.

### FIELD OF INVENTION

The present invention relates to novel heteroaryl aminophenyl ketone derivatives with anti-inflammatory properties, as well as to their inclusion in pharmaceutical compositions and use in therapy, in particular in the treatment of inflammatory diseases.

### BACKGROUND OF THE INVENTION

A series of aminobenzophenones, e.g. 4-(2-amino-4-nitrophenylamino)benzophenone, has been described previously, vide FA Hussein et al., Iraqi J. Sci. 22, 1981, pp. 54-66. In this publication, however, there is no description of any potential therapeutic use of such compounds. WO 98/32730 discloses aminobenzophenone inhibitors of interleukin 1β (IL-1β) and tumour necrosis factor α (TNF-α) secretion in vitro and indicates the potential utility of these compounds in the treatment of inflammatory diseases in which the production of proinflammatory cytokines is involved in the pathogenesis of, for instance, asthma, rheumatoid arthritis, psoriasis, contact dermatitis and atopic dermatitis. Furthermore, the compounds disclosed in WO 98/32730 were tested in vivo for anti-inflammatory properties in the 12-O-tetradecanoylphorbol-13-acetate (TPA) induced murine chronic skin inflammation model (LM DeYoung et al., Agents Actions 26, 1989, pp. 335-341; R P Carlsson et al., Agents Actions 17, 1985, pp. 197-204; I G Alford et al., Agents Actions 37, 1992; P L Stanley et al., Skin Pharmacol. 4, 1991, pp. 262-271). In this chronic skin inflammation model, the compounds had the same potency as the reference compound, hydrocortisone.

It is the object of the present invention to provide pharmacologically active aminophenyl ketone derivatives which differ structurally from those disclosed in WO 98/32730.

### SUMMARY OF THE INVENTION

It has surprisingly been found that novel heteroaryl aminophenyl ketone derivatives are inhibitors of interleukin 1β (IL-1β) and tumour necrosis factor α (TNF-α) secretion in vitro which makes them potentially useful in the treatment and/or prevention of inflammatory diseases and other conditions in which the secretion and modulation of cytokines is involved in the pathogenesis. It has been found that aminophenyl ketone compounds of the present invention exert their anti-inflammatory effect by inhibiting or downregulating MAP kinases, more specifically the p38 MAP kinase, a stress-activated protein which is an important element of the signal transduction pathway leading to the production of pro-inflammatory cytokines.

Accordingly, the present invention relates to a compound of general formula I

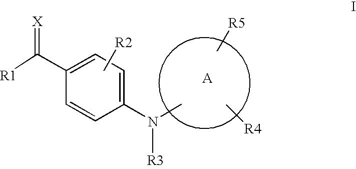

wherein

R₁ is a heteroaromatic ring system comprising 1-4 heteroatoms, optionally substituted by one or more, same or different substituents selected from the group consisting of hydrogen, halogen, haloalkyl, hydroxy, hydroxyalkyl, hydroxyalkyloxy, mercapto, cyano, carboxy, nitro, alkyl, alkenyl, alkynyl, cycloalkyl, cycloalkenyl, heterocycloalkyl, aryl, heteroaryl, aralkyl, alkylaryl, alkoxy, aralkoxy, alkylthio, alkoxycarbonyl, alkylcarbonyloxy, alkoxycarbonyloxy, alkylsulfonyloxy, alkyloxysulfonyl, alkylcarbonylamino, aminocarboaminoalkyl, aminosulfonyl, alkylsulfonylamino, alkanoyl, alkylcarbonyl, —NR₆R₇ or —CONR₆R₇, wherein R₆ and R₇ are the same or different and individually represent hydrogen, alkyl, aryl or —(Z—O)ₙ—Z, wherein Z is alkyl, and n is an integer from 1 to 7;

X is O, S, N—OH or NR₈, wherein R₈ is hydrogen or alkyl;

R₂ is hydrogen, halogen, haloalkyl, hydroxy, hydroxyalkyl, mercapto, cyano, nitro, alkyl, alkenyl, alkynyl, cycloalkyl, aryl, aralkyl, alkylaryl, alkoxy, aralkoxy, alkylthio, alkoxycarbonyl, alkylcarbonylamino, alkylcarbonyloxy, alkoxycarbonyloxy, alkylcarbonyl —NR₆R₇ or —CONR₆R_{7,} wherein R₆ and R₇ are the same or different and individually represent hydrogen, alkyl, aryl or —(Z—O)ₙ—Z;

R₃ is hydrogen, alkyl, alkenyl, alkynyl, cycloalkyl, cycloalkenyl, carboxy or aryl;

A is aryl or a heteroaromatic ring system comprising 1-4 heteroatoms;

R₄ is hydrogen, halogen, haloalkyl, hydroxy, hydroxyalkyl, mercapto, cyano, carboxy, nitro, alkyl, alkenyl, alkynyl, cycloalkyl, heterocycloalkyl, cycloalkenyl, aryl, heteroaryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkyloxysulfonyl, alkylcarbonyl, —N R₆R₇ or —CONR₆R₇, wherein R₆ and R₇ are the same or different and individually represent hydrogen, alkyl, aryl or —(Z—O)ₙ—Z; —N—(C═O)—CF₃, —N—Q—Y, —N—(COO)—Q—Y or —N—(C═O)—N—Q—Y, wherein Q is a bond, —CO—, —CS—, —SO₂—or —CR₉R₁₀—(O—C═O)—, wherein R₉ and R₁₀ are the same or different and individually represent hydrogen, alkyl or aryl, and Y is alkyl, alkenyl, alkynyl, cycloalkyl, cycloalkenyl or aryl, optionally substituted by hydrogen, halogen, haloalkyl, hydroxy, hydroxyalkyl, mercapto, cyano, carboxy, nitro, alkyl, alkenyl, alkynyl, cycloalkyl, cycloalkenyl, aryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkoxysulfonyl, alkylcarbonyl, —NR₆R₇ or —CONR₆R₇, wherein R₆ and R₇ are the same or different and individually represent hydrogen, alkyl or aryl, or —(Z—O)ₙ—Z, wherein Z is alkyl, and n is an integer of from 1 to 7;

R₅ is hydrogen, halogen, haloalkyl, hydroxy, hydroxyalkyl, mercapto, cyano, carboxy, carbamoyl, amino, nitro, alkyl, alkenyl, alkynyl, cycloalkyl, heterocycloalkyl, cycloalkenyl, aryl, heteroaryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkoxysulfonyl, alkylcarbonyl, —NR₆R₇ or —CONR₆R₇, wherein R₆ and R₇ are the same or different and individually represent hydrogen, alkyl, aryl or —(Z—O)ₙ—Z;

and pharmaceutically acceptable salts, hydrates, solvates or esters thereof.

In another aspect, the invention relates to a pharmaceutical composition comprising, as an active component, a compound of formula I together with a pharmaceutically acceptable excipient or carrier.

In another aspect, the invention relates to a compound according to formula I as a medicament.

In a further aspect, the invention relates to the use of a compound of formula I for preparing a medicament for the prevention or treatment of inflammatory diseases or conditions.

In a still further aspect, the invention relates to a method of preventing or treating inflammatory diseases or conditions, the method comprising administering, to a patient in need thereof, an effective amount of a compound of formula I.

### DETAILED DESCRIPTION OF THE INVENTION

Definitions

In the present context, the term “alkyl” is intended to indicate a univalent radical derived from straight or branched alkane by removing a hydrogen atom from any carbon atom. The alkyl chain typically comprises 1-10 carbon atoms, in particular 1-6 carbon atoms. The term includes the subclasses normal alkyl (n-alkyl), secondary and tertiary alkyl, such as methyl, ethyl, n-propyl, isopropyl, n-butyl, isobutyl, sec.-butyl, tert.-butyl, pentyl, isopentyl, hexyl and isohexyl.

The term “haloalkyl” is intended to indicate an alkyl radical as defined above substituted by one or more halogens such as chloro, fluoro, bromo or iodo.

The term “hydroxyalkyl” is intended to indicate an alkyl radical as defined above substituted by one or more hydroxy groups.

The term “alkoxy” is intended to indicate a radical of formula OR′, wherein R′ is alkyl as defined above, e.g. methoxy, ethoxy, propoxy, butoxy, etc.

The term “hydroxyalkyloxy” is intended to indicate an alkoxy group as defined above substituted by one or more hydroxy groups.

The term “alkenyl” is intended to indicate a mono-, di-, tri-, tetra- or pentaunsaturated hydrocarbon radical typically comprising 2-10 carbon atoms, in particular 2-6 carbon atoms, e.g. ethenyl, propenyl, butenyl, pentenyl or hexenyl. The term “alkynyl” is intended to indicate an hydrocarbon radical comprising 1-5 triple C—C bonds, the alkane chain typically comprising 2-10 carbon atoms, in particular 2-6 carbon atoms, such as ethynyl, propynyl, butynyl, pentynyl or hexynyl.

The term “alkoxycarbonyl” is intended to indicate a radical of formula —COOR′ wherein R′ is alkyl as defined above, e.g. methoxycarbonyl, ethoxycarbonyl, n-propoxycarbonyl, isopropoxycarbonyl, etc.

The term “cycloalkyl” is intended to indicate a saturated cycloalkane radical typically comprising 3-10 carbon atoms, in particular 3-8 carbon atoms, e.g. cyclopropyl, cyclobutyl, cyclopentyl or cyclohexyl. The term “cycloalkenyl” is intended to indicate mono-, di- tri- or tetraunsaturated non-aromatic cyclic hydrocarbonsradicals, e.g. cyclopropenyl, cyclobutenyl, cyclopentenyl or cyclohexenyl. The term “heterocycloalkyl” is intended to indicate a cycloalkane radical as defined above, comprising one or more heteroatoms selected from O, N, or S.

The term “aryl” is intended to include radicals of carbocyclic aromatic rings, in particular 5- or 6-membered rings, optionally fused bicyclic rings, e.g. phenyl or naphthyl. The term “heteroaryl” is intended to include radicals of heterocyclic aromatic rings, in particular 5- or 6-membered rings with 1-4 heteroatoms selected from O, S and N, or optionally fused bicyclic rings with 1-4 heteroatoms, e.g. pyridyl, tetrazolyl, thiazolyl, imidazolyl, pyrazolyl, oxazolyl, isoxazolyl, thienyl, pyrazinyl, isothiazolyl, benzimidazolyl and benzofuranyl.

The term “alkylcarbonyloxy” refers to a radical of formula R′—COO—, wherein R′ is alkyl as indicated above. The term “alkoxycarbonyloxy” refers to a radical of formula R′O—COO—, wherein R′ is alkyl as defined above. The term “alkylsulfonyloxy” refers to a radical of formula R′—(SO₂)—O—, wherein R′ is alkyl as defined above. The term “alkyloxysulfonyl” refers to a radical of formula R′O—(SO₂)—, wherein R′ is alkyl as defined above.

The term “aralkyl” is intended to indicate an alkyl radical as defined above comprising an aromatic side chain, e.g. benzyl. The term “alkylaryl” is intended to indicate an aromatic ring with an alkyl side chain as defined above.

The term “halogen” is intended to indicate fluoro, chloro, bromo or iodo.

The term “pharmaceutically acceptable salt” is intended to indicate alkali metal or alkaline earth metal salts, for instance sodium, potassium, magnesium or calcium salts, as well as silver salts and salts with suitable organic or inorganic acids such as hydrochloric, hydrobromic, hydroiodic, sulfuric, nitric, phosphoric, acetic, lactic, maleic, phthalic, citric, propionic, benzoic, glutaric, gluconic, methanesulfonic, salicylic, succinic, tartaric, toluenesulfonic, sulfamic or fumaric acid.

The term “pharmaceutically acceptable esters” is intended to indicate easily hydrolysable esters such as alkanoyloxyalkyl, aralkanoyloxyalkyl, aroyloxyalkyl, e.g. acetoxymethyl, pivaloyloxymethyl, benzoyloxymethyl esters and the corresponding 1′-oxyethyl derivatives, or alkoxycarbonyloxyalkyl esters, e.g. methoxycarbonyloxymethyl esters and ethoxycarbonyloxymethyl esters and the corresponding 1′-oxyethyl derivatives, or lactonyl esters, e.g. phthalidyl esters, or dialkylaminoalkyl esters, e.g. dimethylaminoethyl esters. Easily hydrolysable esters include in vivo hydrolysable esters of the compounds of formula I. Such esters may be prepared by conventional methods known to persons skilled in the art, such as method disclosed in GB patent No. 1 490 852 incorporated herein by reference.

“p38 MAP kinase” is a stress-activated protein kinase existing in several isoforms (p38α, p38β, p38β2, p38γ and p38δ). The p38 MAP kinase is activated by different stimuli including heat, chemical, osmotic, pH and oxidative stress, growth factor withdrawal, high or low glucose and ultraviolet radiation. p38 is also stimulated by agents that mediate the initial physiological response to injury, infection and inflammation, such as LPS and pro-inflammatory cytokines IL-1β, TNF-α, FasL, CD40L and TGF-β. Like other MAP kinases, p38 is phosphorylated by kinases, including MKK3, MEK6 and MKK6, on a threonine and tyrosine in an activation loop (Thr-Xaa-Tyr) close to the ATP and substrate binding site. In turn, p38 phosphorylates and activates the serine-threonine protein kinases MAPKAP kinase-2, MAPKAP kinase-3, MAPKAP kinase-5, MNK-1 and MSK-1. It has been established that activation of p38 regulates cytokine biosynthesis in many cell types either directly by phosphorylating and activating transcription factors involved in the expression of cytokines or indirectly, e.g. by phosphorylating MSK-1 which, when activated, activates the transcription factor CREB. It has also been shown that certain pyridinyl imidazoles, e.g. SB203580, which inhibit p38, inhibit the production of IL-1β and TNF-α from LPS-treated human monocytes. It has therefore been concluded that p38 constitutes a potentially highly interesting target for the development of anti-inflammatory compounds (cf. J C Lee et al., Immunopharmacology 47, 2000, pp. 185-201 and references reviewed therein; P R Young, “Specific Inhibitors of p38 MAP kinase” in Signaling Networks and Cell Cycle Control: The Molecular Basis of Cancer and Other Diseases, J S Gutkind (Ed.), Humana Press, Inc., Totowa, N.J., and references reviewed therein).

Preferred Embodiments of the Compound of Formula I

In compounds of general formula I, R₁ is preferably an optionally substituted, mono- or bicyclic heteroaromatic ring system comprising 1-4 heteroatoms, each ring comprising 5 or 6 ring atoms. Examples of suitable heteroaromatic ring systems are selected from the group consisting of thienyl, furyl, benzofuranyl, isobenzofuranyl, pyrrolyl, imidazolyl, benzimidazolyl, pyrazolyl, thiazolyl, isothiazolyl, isoxazolyl, pyridyl, pyrazinyl, pyrimidinyl, pyridazinyl, indolizinyl, isoindolyl, indolyl, indazolyl, purinyl, quinolyl, isoquinolyl, phthalazinyl, quinazolinyl, benzothienyl, isobenzothienyl, benzothiazolyl, benzoisothiazolyl, triazolyl, tetrazolyl, quinoxalyl, allopurinyl, benzotriazolyl or oxazolyl.

In a particularly favoured embodiment, R₁ is a heteroaromatic ring system comprising one heteroatom, such as thienyl, furyl, pyrrolyl, pyridyl, benzofuranyl, benzothienyl, indolyl, isoindolyl, quinolyl, isoquinolyl, isobenzothienyl or isobenzofuryl.

The heteroatom of the heteroaromatic ring system is preferably nitrogen or sulphur, such as thienyl, benzothienyl, isobenzothienyl and pyridyl.

The heteroaromatic ring system R₁ may suitably be substituted by one or more of the same or different substituents which are preferably selected from the group consisting of halogen, hydroxy, hydroxyalkyl, hydroxyalkyloxy, trifluoromethyl, C₁₋₄ alkyl, C₂₋₄ alkenyl, C₂₋₄ alkynyl, C₁₋₄ alkoxycarbonyl, cyano, —NR₆R₇ or CONR₆R_{7,} wherein R6 and R₇ are as indicated above.

In preferred compounds of general formula I, R₂ may suitably be hydrogen, hydroxy, halogen, mercapto, trifluoromethyl, amino, C₁₋₆ alkyl, C₂₋₆ alkenyl, C₂₋₆ alkynyl, C₁₋₆ alkoxy C₁₋₆ alkylthio, C₁₋₆ alkylamino, C₁₋₆ alkoxycarbonyl, cyano, —CONH₂, aryl or nitro, in particular hydrogen, halogen, hydroxy, trifluoromethyl, amino, C₁₋₄ alkyl, C₂₋₄ alkenyl or C₁₋₄ alkoxy.

In preferred compounds of general formula I, R₃ may suitably be hydrogen, C₁₋₆ alkyl, C₁₋₆ alkenyl or aryl.

In preferred compounds of general formula I, R₄ may suitably be halogen, CN, CF₃, C₁₋₆ alkyl, —NH₂ or —N—(C═O)—CF₃. In other preferred compounds of formula I, R₄ may suitably be —N—Q—Y, wherein Q is a bond —CO— or —CS—, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkyl or aryl. Alternatively, in compounds of formula I, R₄ may be —N—(COO)—Q—Y, wherein Q is a bond or —CR₉R₁₀—(O—C═O)—, wherein R₉ and R₁₀ are the same or different and individually represent hydrogen, trifluoromethyl or C₁₋₆ alkyl, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkenyl or aryl. As a further alternative embodiment, R₄ may be —N—(C═O)—N—Q—Y, wherein Q is a bond or —CR₉R₁₀—(O—C═O)—, wherein R₉ and R₁₀ are the same or different and individually represent hydrogen, trifluoromethyl or C₁₋₆ alkyl, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkenyl, aryl or —(Z—O)ₙ—Z, wherein Z and n are as indicated above.

In preferred compounds of formula I, R₅ is suitably selected from hydrogen, halogen, hydroxy, mercapto, trifluoromethyl, amino, C₁₋₆ alkyl, C₂₋₆ alkenyl, C₂₋₆ alkynyl, C₁₋₆ alkoxy, C₁₋₆ alkylthio, C₁₋₆ alkylamino, C₁₋₆ alkoxycarbonyl, aryl, cyano, carboxy or carbamoyl.

In preferred compounds of formula I, X is O.

In preferred compounds of formula I, A is phenyl.

Accordingly, currently preferred compounds of the invention are compounds of the general formula Ia

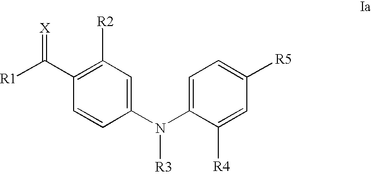

wherein R₁, R₂, R₃, R_{4,} R₅ and X are as indicated above.

Examples of specific compounds of the invention wherein X is O are

4-(2-tolylamino)-2-chlorophenyl 3-chloro-2-thienyl ketone (compound 101)

4-(2-nitrophenylamino)-2-chlorophenyl 3-chloro-2-thienyl ketone (compound 102)

4-(2-aminophenylamino)-2-chlorophenyl 3-chloro-2-thienyl ketone (compound 103)

4-(2-tolylamino)-2-chlorophenyl 3-methyl-2-thienyl ketone (compound 106)

4-(2-nitrophenylamino)-2-chlorophenyl 3-methyl-2-thienyl ketone (compound 107)

4-(2-aminophenylamino)-2-chlorophenyl 3-methyl-2-thienyl ketone (compound 108)

4-(2-nitrophenylamino)-2-chlorophenyl 1-methyl-2-pyrrolyl ketone (compound 111)

4-(2-aminophenylamino)-2-chlorophenyl 1-methyl-2-pyrrolyl ketone (compound 112)

4-(2-tolylamino)-2-chlorophenyl 1-methyl-2-pyrrolyl ketone (compound 113)

4-(2-tolylamino)-2-chlorophenyl 1,3,5-trimethyl-4-pyrazolyl ketone (compound 114)

3-methyl-2-benzothienyl 4-(2-tolylamino)-2-chlorophenyl ketone (compound 115)

4-(2-tolylamino)-2-chlorophenyl 4-methyl-3-thienyl ketone (compound 116)

4-(2-tolylamino)-2-chlorophenyl 2,5-dimethyl-3-thienyl ketone (compound 117)

3-methyl-2-furyl 4-(2-tolylamino)-2-chlorophenyl ketone (compound 118)

5-methyl-2-furyl 4-(2-tolylamino)-2-chlorophenyl ketone (compound 121)

4-(2-tolylamino)-2-chlorophenyl 2-pyridyl ketone (compound 122)

4-(2-tolylamino)-2-chlorophenyl 3-chloro-2-pyridyl ketone (compound 123)

1-methyl-2-imidazolyl 4-(2-tolylamino)-2-chlorophenyl ketone (compound 126)

1-methyl-2-imidazolyl 4-(2-nitrophenyl)amino-2-chlorophenyl ketone (compound 127)

1-methyl-2-imidazolyl 4-(2-aminophenylamino)-2-chlorophenyl ketone (compound 128)

3,5-dimethyl-4-isoxazolyl 4-(2-tolylamino)-2-chlorophenyl ketone (compound 129)

4-(2-tolylamino)-2-chlorophenyl 4,5-dimethyl-2-thiazolyl ketone (compound 130)

4-(2-tolylamino)-2-chlorophenyl 5-methyl-2-thienyl ketone (compound 131)

4-(2-benzonitrileamino)-2-chlorophenyl 3-chloro-2-thienyl ketone (compound 132)

4-(2-trifluoromethylphenylamino)-2-chlorophenyl 3-chloro-2-thienyl ketone (compound 133)

4-(4-bromo-2-tolylamino)-2-chlorophenyl 3-chloro-2-thienyl ketone (compound 134)

4-(4-bromo-2-cyanophenylamino)-2-chlorophenyl 3-chloro-2-thienyl ketone (compound 135)

4-(4-isoquinolylamino)-2-chlorophenyl 3-chloro-2-thienyl ketone (compound 136)

4-(4-bromo-2,3,5,6-tetramethylphenylamino)-2-chlorophenyl 3-chloro-2-thienyl ketone (compound 137)

4-(4-bromo-2-chlorophenylamino)-2-chlorophenyl 3-chloro-2-thienyl ketone (compound 138)

4-(4-bromo-2-nitrophenylamino)-2-chlorophenyl 3-chloro-2-thienyl ketone (compound 139)

4-(2-amino-4-bromophenylamino)-2-chlorophenyl 3-chloro-2-thienyl ketone (compound 140)

4-phenylamino-2-chlorophenyl 3-chloro-2-thienyl ketone (compound 141)

4-(4-bromo-2-trifluoromethylphenylamino)-2-chlorophenyl 3-chloro-2-thienyl ketone (compound 142)

4-(3-pyridylamino)-2-chlorophenyl 3-chloro-2-thienyl ketone (compound 143)

1-[4-(2-tolylamino)-2-chlorobenzoyl]pyrrole (Compound 146)

4-(2-tolylamino)-2-chlorophenyl 3,5-dimethyl-2-pyrrolyl ketone (compound 149)

4-(2-amino-4-trifluoromethylphenylamino)-2-chlorophenyl 3-chloro-2-thienyl ketone (compound 150)

4-(2-tolylamino)-2-chlorophenyl 3-chloro-4-pyridyl ketone (compound 151)

5-chloro-3-methyl-2-benzothienyl 4-(2-tolylamino)-2-chlorophenyl ketone (compound 152)

2-benzofuranyl 4-(2-tolylamino)-2-chlorophenyl ketone (Compound 153)

3-benzothienyl 4-(2-tolylamino)-2-chlorophenyl ketone (Compound 154)

3,5-dimethyl-2-benzothienyl 4-(2-tolylamino)-2-chlorophenyl ketone (Compound 155)

4-(2-tolylamino)-2-chlorophenyl 3-chloro-2-pyrazinyl ketone (Compound 156)

1-[4-(2-tolylamino)-2-chlorobenzoyl]indole (Compound 159)

4-(2-tolylamino)-2-chlorophenyl 2,5-dichloro-3-thienyl ketone (Compound 160)

5-indolyl 4-(2-tolylamino)-2-chlorophenyl ketone (Compound 161)

4-(2-tolylamino)-2-chlorophenyl 3-chloro-1-oxidopyridin-2-yl ketone (Compound 162)

4-[methyl(2-tolyl)amino]-2-chlorophenyl 3-chloro-2-thienyl ketone (Compound 163)

Specific compounds wherein X is S are

4-(2-tolylamino)-2-chlorophenyl 3-chloro-2-thienyl thioketone

4-(2-nitrophenylamino)-2-chlorophenyl 3-chloro-2-thienyl thioketone

4-(2-aminophenylamino)-2-chlorophenyl 3-chloro-2-thienyl thioketone

4-(2-tolylamino)-2-chlorophenyl 3-methyl-2-thienyl thioketone

4-(2-nitrophenylamino)-2-chlorophenyl 3-methyl-2-thienyl thioketone

4-(2-aminophenylamino)-2-chlorophenyl 3-methyl-2-thienyl thioketone

4-(2-nitrophenylamino)-2-chlorophenyl 1-methyl-2-pyrrolyl thioketone

4-(2-aminophenylamino)-2-chlorophenyl 1-methyl-2-pyrrolyl thioketone

4-(2-tolylamino)-2-chlorophenyl 1-methyl-2-pyrrolyl thioketone

4-(2-tolylamino)-2-chlorophenyl 1,3,5-trimethyl-4-pyrazolyl thioketone

3-methyl-2-benzothienyl 4-(2-tolylamino)-2-chlorophenyl thioketone

4-(2-tolylamino)-2-chlorophenyl 4-methyl-3-thienyl thioketone

4-(2-tolylamino)-2-chlorophenyl 2,5-dimethyl-3-thienyl thioketone

3-methyl-2-furyl 4-(2-tolylamino)-2-chlorophenyl thioketone

5-methyl-2-furyl 4-(2-tolylamino)-2-chlorophenyl thioketone

4-(2-toylylamino)-2-chlorophenyl 2-pyridyl thioketone

4-(2-tolylamino)-2-chlorophenyl 3-chloro-2-pyridyl thioketone

1-methyl-2-imidazolyl 4-(2-tolylamino)-2-chlorophenyl thioketone

1-methyl-2-imidazolyl 4-(2-nitrophenyl)amino-2-chlorophenyl thioketone

1-methyl-2-imidazolyl 4-(2-aminophenylamino)-2-chlorophenyl thioketone

3,5-dimethyl -4-isoxazolyl 4-(2-tolylamino)-2-chlorophenyl thioketone

4-(2-tolylamino)-2-chlorophenyl 4,5-dimethyl-2-thiazolyl thioketone

4-(2-tolylamino)-2-chlorophenyl 5-methyl-2-thienyl thioketone

4-(2-benzonitrileamino)-2-chlorophenyl 3-chloro-2-thienyl thioketone

4-(2-trifluoromethylphenylamino)-2-chlorophenyl 3-chloro-2-thienyl thioketone

4-(4-bromo-2-tolylamino)-2-chlorophenyl 3-chloro-2-thienyl thioketone

4-(4-bromo-2-cyanophenylamino)-2-chlorophenyl 3-chloro-2-thienyl thioketone

4-(4-isoquinolylamino)-2-chlorophenyl 3-chloro-2-thienyl thioketone

4-(4-bromo-2,3,5,6-tetramethylphenylamino)-2-chlorophenyl 3-chloro-2-thienyl thioketone

4-(4-bromo-2-chlorophenylamino)-2-chlorophenyl l 3-chloro-2-thienyl thioketone

4-(4-bromo-2-nitrophenylamino)-2-chlorophenyl 3-chloro-2-thienyl thioketone

4-(2-amino-4-bromophenylamino)-2-chlorophenyl 3-chloro-2-thienyl thioketone

4-phenylamino-2-chlorophenyl 3-chloro-2-thienyl thioketone

4-(4-bromo-2-trifluoromethylphenylamino)-2-chlorophenyl 3-chloro -2-thienyl thioketone

4-(3-pyridylamino)-2-chlorophenyl 3-chloro-2-thienyl thioketone

1-[4-(2-tolylamino)-2-chlorothiobenzoyl]pyrrole

4-(2-tolylamino)-2-chlorophenyl 3,5-dimethyl-2-pyrrolyl thioketone

4-(2-amino-4-trifluoromethylphenylamino)-2-chlorophenyl 3-chloro-2-thienyl thioketone

4-(2-tolylamino)-2-chlorophenyl 3-chloro-4-pyridyl thioketone

5-chloro-3-methyl-2-benzothienyl 4-(2-tolylamino)-2-chlorophenyl thioketone

2-benzofuranyl 4-(2-tolylamino)-2-chlorophenyl thioketone

3-benzothienyl 4-(2-tolylamino)-2-chlorophenyl thioketone

3,5-dimethyl-2-benzothienyl 4-(2-tolylamino)-2-chlorophenyl thioketone

4-(2-tolylamino)-2-chlorophenyl 3-chloro-2-pyrazinyl thioketone

1-[4-(2-tolylamino)-2-chlorothiobenzoyl]indole

4-(2-tolylamino)-2-chlorophenyl 2,5-dichloro-3-thienyl thioketone

5-indolyl 4-(2-tolylamino)-2-chlorophenyl thioketone

4-(2-tolylamino)-2-chlorophenyl 3-chloro-1-oxidopyridin-2-yl thioketone

4-[methyl(2-tolyl)amino]-2-chlorophenyl 3-chloro-2-thienyl thioketone

Specific compounds wherein X is N—OH are

4-(2-tolylamino)-2-chlorophenyl 3-chloro-2-thienyl ketoxime

4-(2-nitropenylamino)-2-chlorophenyl 3-choro-2-thienyl ketoxime

4-(2-aminophenylamino)-2-chlorophenyl 3-chloro-2-thienyl ketoxime

4-(2-tolylamino)-2-chlorophenyl 3-methyl-2-thienyl ketoxime

4-(2-nitrophenylamino)-2-chlorophenyl 3-methyl-2-thienyl ketoxime

4-(2-aminophenylamino)-2-chlorophenyl 3-methyl -2-thienyl ketoxime

4-(2-nitrophenylamino)-2-chlorophenyl 1-methyl-2-pyrrolyl ketoxime

4-(2-aminophenylamino)-2-chlorophenyl 1-methyl-2-pyrrolyl ketoxime

4-(2-tolylamino)-2-chlorophenyl 1-methyl-2-pyrrolyl ketoxime

4-(2-tolylamino)-2-chlorophenyl 1,3,5-trimethyl-4-pyrazolyl ketoxime

3-methyl-2-benzothienyl 4-(2-tolylamino)-2-chlorophenyl ketoxime

4-(2-tolylamino)-2-chlorophenyl 4-methyl-3-thienyl ketoxime

4-(2-tolylamino)-2-chlorophenyl 2,5-dimethyl-3-thienyl ketoxime

3-methyl-2-furyl 4-(2--tolylamino)-2-chlorophenyl ketoxime

5-methyl-2-furyl 4-(2-tolylamino)-2-chlorophenyl ketoxime

4-(2-tolylamino)-2-chlorophenyl 2-pyridyl ketoxime

4-(2-tolylamino)-2-chlorophenyl 3-chloro-2-pyridyl ketoxime

1-methyl-2-imidazolyl 4-(2-tolylamino)-2-chlorophenyl ketoxime

1-methyl-2-imidazolyl 4-(2-nitrophenyl)amino-2-chlorophenyl ketoxime

1-methyl-2-imidazolyl 4-(2-aminophenylamino)-2-chlorophenyl ketoxime

3-5-dimethyl -4-isoxazolyl 4-(2-tolylamino)-2-chlorophenyl ketoxime

4-(2-tolylamino)-2-chlorophenyl 4,5-dimethyl-2-thiazolyl ketoxime

4-(2-tolylamino)-2-chlorophenyl 5-methyl-2-thienyl ketoxime

4-(2-benzonitrileamino)-2-chlorophenyl 3-chloro-2-thienyl ketoxime

4-(2-trifluoromethylphenylamino)-2-chlorophenyl 3-chloro-2-thienyl ketoxime

4-(4-bromo-2-tolylamino)-2-chlorophenyl 3-chloro-2-thienyl ketoxime

4-(4-bromo-2-cyanophenylamino)-2-chlorophenyl 3-chloro-2-thienyl ketoxime

4-(4-isoquinolylamino)-2-chlorophenyl 3-chloro-2-thienyl ketoxime

4-(4-bromo-2,3,5,6-tetramethylphenylamino)-2-chlorophenyl 3-chloro-2-thienyl ketoxime

4-(4-bromo-2-chlorophenylamino)-2-chlorophenyl 3-chloro-2-thienyl ketoxime

4-(4-bromo-2-nitrophenylamino)-2-chlorophenyl 3-chloro-2-thienyl ketoxime

4-(2-amino-4-bromophenylamino)-2-chlorophenyl 3-chloro-2-thienyl ketoxime

4-phenylamino-2-chlorophenyl 3-chloro-2-thienyl ketoxime

4-(4-bromo-2-trifluoromethylphenylamino)-2-chlorophenyl 3-chloro-2-thienyl ketoxime

4-(3-pyridylamino)-2-chlorophenyl 3-chloro-2-thienyl ketoxime

1-[4-(2-tolylamino)-2-chlorobenzoxime]pyrrole

4-(2-tolylamino)-2-chlorophenyl 3,5-dimethyl-2-pyrrolyl ketoxime

4-(2-amino-4-trifluoromethylphenylamino)-2-chlorophenyl 3-chloro-2-thienyl ketoxime

4-(2-tolylamino)-2-chlorophenyl 3-chloro-4-pyridyl ketoxime

5-chloro-3-methyl-2-benzothienyl 4-(2-tolylamino)-2-chlorophenyl ketoxime

2-benzofuranyl 4-(2-tolylamino)-2-chlorophenyl ketoxime

3-benzothienyl 4-(2-tolylamino)-2-chlorophenyl ketoxime

3,5-dimethyl-2-benzothienyl 4-(2-tolylamino)-2-chlorophenyl ketoxime

4-(2-tolylamino)-2-chlorophenyl 3-chloro-2-pyrazinyl ketoxime

1-[4-(2-tolylamino)-2-chlorobenzoxime]indole

4-(2-tolylamino)-2-chlorophenyl 2,5-dichloro-3-thienyl ketoxime

5-indolyl 4-(2-tolylamino)-2-chlorophenyl ketoxime

4-(2-tolylamino)-2-chlorophenyl 3-chloro-1-oxidopyridin-2-yl ketoxime

4-[methyl(2-tolyl)amino]-2-chlorophenyl 3-chloro-2-thienyl ketoxime

Compounds of the present invention may comprise one or more asymmetric carbon atoms or carbon-carbon double bonds which allow for stereo and geometric isomeric forms. It is to be understood that the present invention relates to all such forms, in pure form or as mixtures thereof. If the compounds are synthesised as a mixture of isomeric forms it is possible to separate the mixture into its isomeric forms. It lies within the capability of a person skilled in the art to make such separation, and methods available to him include chromatography, e.g. chiral chromatography. It may also be possible to synthesise pure isomeric forms directly provided the starting materials are pure isomers, too.

Methods of Preparing Compounds of Formula I and Ia

The compounds of the present invention may be prepared in a number of ways well known to those skilled in the art of organic synthesis. The compounds of the present invention may be synthesised using the methods outlined below, together with methods generally known in the art of synthetic organic chemistry, or variations thereof as appreciated by those skilled in the art. Preferred methods include, but are not limited to, those described below.

The novel compounds of formula I and Ia may be prepared using the reactions and techniques described in this section. The reactions are performed in solvents appropriate to the reagents and materials employed and are suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis that the functionality present on various portions of the educt molecule must be compatible with the reagents and reactions proposed. Not all compounds of formula I falling into a given class may be compatible with some of the reaction conditions required in some of the methods described. Such restrictions to the substituents which are compatible with the reaction conditions will be readily apparent to one skilled in the art and alternate methods can be used.

The following abbreviations have been used throughout this specification: BINAP=racemic or non-racemic 2,2′-bis(diphenylphosphino)-1,1′-binaphthyl, CDCl₃=deuteriochloroform, DMF=N,N-dimethyl-formamide, DMSO-d₆=hexadeuterodimethylsulfoxide, DMSO=dimethylsulfoxide, EtOAc=ethyl acetate, Et₂O=diethylether, Pd₂(dba)₃=tris(dibenzylideneacetone)dipalladium(O), MeOH=methanol, NaOt-Bu=sodium-tert-butoxide, KOt-Bu=potassium-tert-butoxide, THF=tetrahydrofuran, PE=petroleumsether (bp 40-60° C.), TLC=thin layer chromatography.

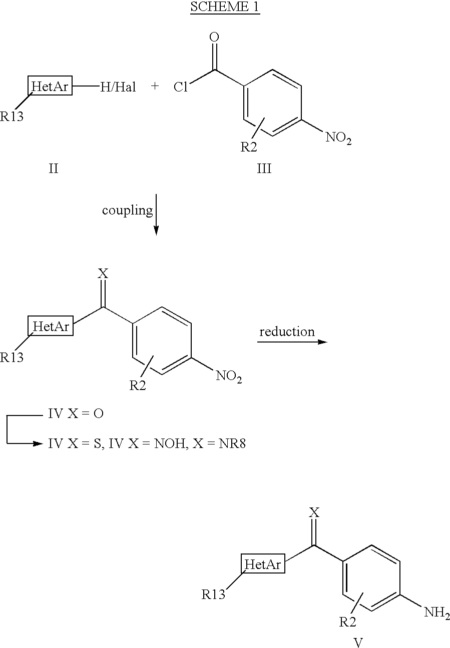

R₁₃ is a substituent of R₁ as defined in general formula I

HetAr=heteroaromatic ring system

Compounds according to the present invention with the general formula V may be prepared by several methods known to those skilled in the art of organic synthesis. One useful sequence is shown in Scheme 1. The key step comprising coupling of a protio-, a bromo- (or iodo-) heteroaryl with the general formula II with an acid chloride with the general formula III to afford the heteroaryl phenylketone with the general formula IV. This compound IV may then be reduced to the corresponding amine with the general formula V by treatment with standard reducing agents. Examples of such reducing agents include, but are not limited to, stannous chloride dihydrate, hydrogen, ammonium formiate, or hydrazine hydrate and a catalytic amount of palladium on carbon. The coupling reaction is done by transforming II into a reactive organometallic intermediate, e.g. by treatment with butyllithium to afford the lithium derivative or by treatment with iso-propylmagnesium chloride to afford the magnesium derivative. This intermediate is then reacted with the acid chloride, with the general formula III.

Compounds according to the present invention may in special cases be prepared by a simple functional group interconversion (FGI), meaning a standard process, known to those skilled in the art of organic synthesis, where a functional group in compounds with the general formula I is transformed into a different functional group in one or more synthetic steps, leading to a new compound with the general formula I. Examples of such processes are, but are not limited to, hydrolysis of an ester to give an acid under basic conditions; deprotection of a methylether to give a phenol by treatment with e.g. borontribromide (BBr₃); and catalytic hydrogenation of an olefin to give a saturated hydrocarbon.

Compounds according to the present invention with the general formula I where X=S may be prepared from the ketone (with the general formula I, X=O) by such a FGI process, by using one of the many thiocarbonylating reagent, known to those skilled in the art of organic synthesis. Examples of such thiocarbonylating reagents include, but are not limited to, phosphorous pentasulfide (P₄S₁₀), or Lawesson's reagent (2,4-bis(4-methoxyphenyl)-1,3,2,4-dithiaphosphetane-2,4-disulfide) or the like.

Compounds according to the present invention with the general formula I where X=N—OH may be prepared from the ketone (with the general formula I, X=O) by treatment with hydroxylamine, or a protected derivative thereof followed by deprotection, in an appropriate solvent like e.g. pyridine or methanol.

Compounds according to the present invention with the general formula I where X=N—R8 may be prepared from the ketone (with the general formula I, X=O) by treatment with a primary amine.

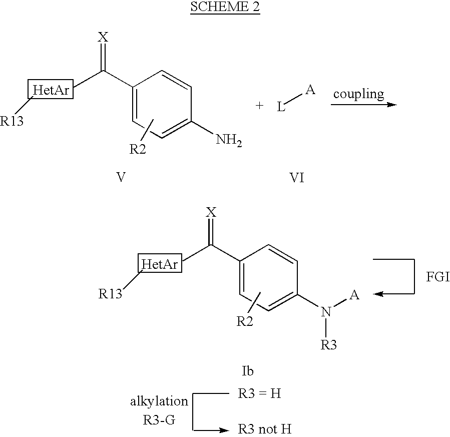

L: Leaving group, such as F, Cl, Br, I, or OSO₂CF₃

G: Leaving group, such as Cl, Br, I, OSO₂CF₃, or OTs

FGI: Functional group interconversion

Compounds according to the present invention may be prepared by a process comprising coupling of an amine of the formula V with a bromide, iodide, fluoride, chloride or triflate with the formula VI, as shown in Scheme 2, wherein R₁₃ is as defined in Scheme 1 and R₂, R₃ and X are as defined in general formula I, except that any substituents or functional group which are potentially reactive in the coupling reaction may themselves be protected before the coupling reaction is performed and the perotection subsequently subsequently removed.

The coupling reaction is carried out using any of the methods for the formation of diphenylamines known to one skilled in the art of organic synthesis.

The preferred method is the palladium catalysed amination method which comprises coupling of an amine with an (hetero)arylhalogenide (or (hetero)aryltriflate) in the presence of a base, a suitable Pd source, and a suitable phosphine ligand in an inert solvent.

Many palladium compound may be used in the process is not particularly limited, and specific examples include palladium(II) acetate, palladium(II) chloride, palladium(II) bromide, dichlorobis(triphenylphosphine)palladium(II), tetrakis(triphenylphosphine)palladium(0), tris(dibenzylideneacetone)dipalladium(0). The prefered ligand include, but are not limited to, racemic or non-racemic 2,2′-bis(diphenylphosphino)-1,1′-binaphthyl (hereinafter refered to as BINAP), tri-o-tolylphosphine, tri-tert-butylphosphine, 1,1′-bis(diphenylphosphino)-ferrocene, bis[(2-diphenylphosphino)phenyl]ether (DPEphos), 2-dicyclohexylphosphanyl-2′-dimethylaminobiphenyl, 2-(di-tert-butylphosphino)biphenyl, and 9,9-dimethyl-4,6-bis(diphenylphosphino)xanthene (Xantphos). The amount of palladium and ligand used in this process is typically in the range 0.1 to 10% by mole relative to the amount of the aromatic halide (or triflate) used.

Especially NaOt-Bu and cesium carbonate (Cs₂CO₃) have proven to be the best bases in this process, but other bases may be used as well.

The reaction is typically performed at elevated temperature (80-120° C.) in inert solvents like 1,4-dioxane, toluene, benzene and tetrahydrofuran under an inert atmosphere like argon or nitrogen.

The coupling reaction may also be carried out by nucleophilic substitution of a (hetero)arylhalogenide with an amine, either in the presence of a base in a polar aprotic solvent, or without a solvent.

The preferred base is KOt-Bu or NaH, but other bases may be used as well.

The preferred solvent is dimethyl sulfoxide, but other solvents such as DMF may be used as well.

The reaction is carried out at room temperature for 12-48 h.

Compounds according to the present invention in which R₃ is not hydrogen may be prepared by a process comprising coupling of an amine of the formula I (R₃=H) with an alkylating agent, as shown in Scheme 2, where R₁₃ is as defined in scheme 1, and where R₂, R₃, A and X are as defined in general formula I, except that any substituents or functional group which are potentially reactive in the coupling reaction may themselves be protected before the coupling reaction is performed and subsequently removed.

Typically alkylating agents of the general formula R3-G include, but are not limited to, iodides (G=I), bromides (G=Br), chlorides (G=Cl) and sulfonates (G=OSO₂R′, where R′ represents methyl, trifluoromethyl or 4-methylphenyl).

An alternative process for the preparation of compounds with the general formula I may be through the synthesis of compounds of general formula VIII as shown in scheme 3. Compounds of general formula VIII may then be coupled with compounds of general formula II as described in scheme 1.

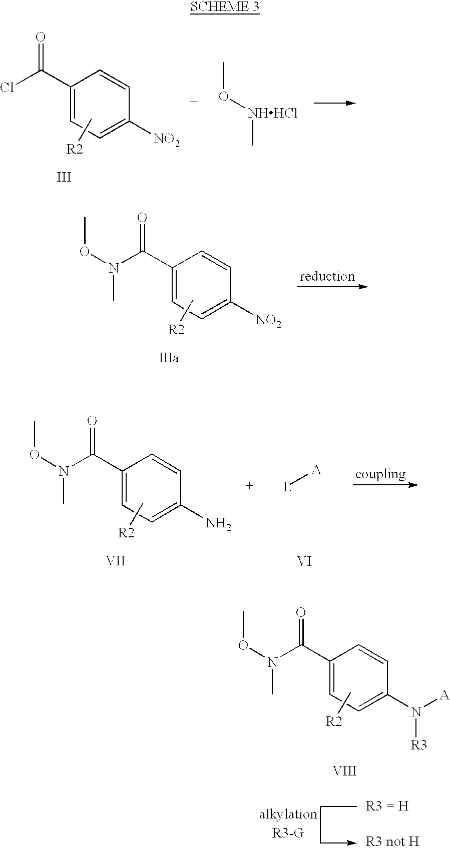

The FGI from the acid chloride of general formula III to the N-methoxy-N-methylbenzamide IIIa is following methods known to those skilled in the art of organic synthesis. Reduction to compounds of general formula VII may be performed as described above (scheme 1). Coupling affording compounds of general formula VIII may be performed as described above (scheme 2). After appropriate protection of VIII (R3 =H) with for example various silyl groups (R3 =Si(alkyl)₃), VIII may be coupled with compounds of general formula II as described for scheme 1 affording directly compounds of general formula I or a protected derivative thereof.

As shown in Scheme 1 compounds with the general formula IV (X=O) may be transformed by a FGI process to give compounds with the general formula IV (X=S or X=N—OH or X=N—R₈) as described above. This is only to illustrate the flexibility in the synthesis and in general the described sequence of processes is only one of many possible strategies for the synthesis of compound of the present invention. That is, it may be more advantageous in some cases to alter the sequence of the processes described above. The described sequence of processes is not considered as being limited for the preparation of the compounds of the present invention with the general formula I and alteration of the reaction sequence is an obvious alternative for those skilled in the art of organic synthesis.

Pharmaceutical Compositions

In another aspect, the invention relates to a pharmaceutical composition comprising, as an active component, a compound of formula I, optionally together with a pharmaceutically acceptable excipient or diluent. Furthermore, the invention relates to the use of a compound of formula I for the preparation of a medicament for the prevention or treatment of inflammatory diseases or conditions.

Pharmaceutical compositions of the invention may be in unit dosage form such as tablets, pills, capsules, powders, granules, elixirs, syrups, emulsions, ampoules, suppositories or parenteral solutions or suspensions; for oral, parenteral, opthalmic, transdermal, intra-articular, topical, pulmonal, nasal, buccal or rectal administration or in any other manner appropriate for the formulation of anti-inflammatory compounds and in accordance with accepted practices such as those disclosed in Remington: The Science and Practice of Pharmacy. 19^{th} Ed., Mack Publishing Company, 1995. In the composition of the invention, the active component may be present in an amount of from about 0.1-100% by weight of the composition.

For oral administration in the form of a tablet or capsule, a compound of formula I may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum or the like.

For the preparation of solid compositions such as tablets, the active compound of formula I is mixed with one or more excipients, such as the ones described above, and other pharmaceutical diluents such as water to make a solid preformulation composition containing a homogenous mixture of a compound of formula I. The term “homogenous” is understood to mean that the compound of formula I is dispersed evenly throughout the composition so that the composition may readily be subdivided into equally effective unit dosage forms such as tablets or capsules. The preformulation composition may then be subdivided into unit dosage forms containing from about 0.05 to about 1000 mg, in particular from about 0.1 to about 500 mg, of the active compound of the invention.

Liquid formulations for either oral or parenteral administration of the compound of the invention include, e.g., aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, e.g. intramuscular, intraperitoneal, subcutaneous or intravenous injection or infusion, the pharmaceutical composition preferably comprises a compound of formula I dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances. The composition may be sterilised by, for instance, filtration through a bacteria-retaining filter, addition of a sterilising agent to the composition, irradiation of the composition, or heating the composition. Alternatively, the compound of the invention may be provided as a sterile, solid preparation, e.g. a freeze-dried powder, which is dissolved in sterile solvent immediately prior to use.

The composition intended for parenteral administration may additionally comprise conventional additives such as stabilisers, buffers or preservatives, e.g. antioxidants such as methylhydroxybenzoate or the like.

Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. For topical administration, the compound of formula I may typically be present in an amount of from 1 to 20% by weight of the composition, but may also be present in an amount of up to about 50% of the composition.

Compositions suitable for administration to the nasal or buccal cavity or for inhalation include powder, self-propelling and spray formulations, such as aerosols and atomizers. Such compositions may comprise a compound of formula I in an amount of 0.1-20%, e.g. 2%, by weight of the composition.

The composition may additionally comprise one or more other active components conventionally used in the treatment of various inflammatory diseases and conditions. Examples of such additional active components may be selected from the group consisting of glucocorticoids, vitamin D and vitamin D analogues, antihistamines, platelet activating factor (PAF) antagonists, anticholinergic agents, methylxanthines, β-adrenergic agents, COX-2 inhibitors, salicylates, infomethacin, flufenamate, naproxen, timegadine, gold salts, penicillamine, serum cholesterol lowering agents, retinoids, zinc salts and salicylazosulfapyridine.

In a further aspect, the invention relates to a method of treating inflammatory diseases or conditions, the method comprising administering, to a patient in need thereof, an effective amount of a compound of formula I.

A suitable dosage of the compound of the invention will depend, inter alia, on the age and condition of the patient, the severity of the disease to be treated and other factors well known to the practising physician. The compound may be administered either orally or parenterally according to different dosing schedules, e.g. daily or with weekly intervals. In general a single dose will be in the range from 0.1 to 400 mg/kg body weight. The compound may be administered as a bolus (i.e. the entire daily dosis is administered at once) or in divided doses two or more times a day.

Inflammatory diseases or conditions contemplated for treatment with the present compounds are inflammatory diseases where modulation of cytokine expression and secretion may be mediated by MAP kinases such as the p38 MAP kinase as discussed above. Examples of inflammatory diseases or conditions believed to be mediated by the p38 MAP kinase are selected from the group consisting of asthma, arthritis, including rheumatoid arthritis and spondyloarthritis, gout, atherosclerosis, inflammatory bowel disease, Crohn's disease, proliferative and inflammatory skin disorders, such as psoriasis, atopic dermatitis and acne vulgaris, uveitis, sepsis, septic shock and osteoporosis.

The treatment may additionally involve administration of one or more other anti-inflammatory active components such as glucocorticoids, vitamin D and vitamin D analogues, antihistamines, platelet activating factor (PAF) antagonists, anticholinergic agents, methylxanthines, β-adrenergic agents, COX-2 inhibitors, salicylates, indomethacin, flufenamate, naproxen, timegadine, gold salts, penicillamine, serum cholesterol lowering agents, retinoids, zinc salts and salicylazosulfapyridine. The administration of a compound of the present invention and another anti-inflammatory component may be either concomitantly or sequentially.

Pharmacological Methods

To study the effect of compounds of the present invention in vitro the inhibition of the IL-1β and TNF-α secretion was measured using the following procedure:

Cytokine production was measured in the media from lipopolysaccharide (LPS) stimulated peripheral blood mononuclear cells. The mononuclear cells were isolated from human peripheral blood by Lymphoprep® (Nycomed, Norway) fractionation and suspended in RPMI 1640 (growth medium) with foetal calf serum (FCS, 2%), at a concentration of 5×10⁵ cells/ml. The cells were incubated in 24-well tissue culture plates in 1 ml aliquots. Test compounds were dissolved in dimethylsulfoxide (DMSO, 10 mM) and were diluted with the medium. Compounds were added to the cells for 30 minutes, then LPS (1 mg/ml final concentration) was added. The plates were incubated for 18 hours, and the concentration of IL-1β and TNF-α in the medium was determined by enzyme-linked immunosorbent assays. The median inhibitory concentrations (IC₅₀) of the compounds were calculated. The results are shown in Table 1.

The compounds of the present invention also show similar activities in the ability to inhibit PMN (polymorphonuclear) superoxide secretion which is also indicative of potentially useful anti-inflammatory drugs. The compounds were tested using the following procedure:

Human polymorphonuclear (PMN) granulocytes were isolated from human blood by dextran sedimentation, Lymphoprep® fractionation and hypotonic lysis of contaminating erythrocytes.

Superoxide anion generation was measured as the superoxide dismutase inhibitable reduction of ferricytochrome C (Madhu, S.B. et al, Inflammation, 16, 241, (1992)). The cells were suspended in Hanks' balanced salt solution, and incubated for 10 minutes at 37° C. with test compounds. The cells were primed by the addition of TNF-α (3 ng/ml final concentration) for 10 minutes, and then ferricytochrome C, (final concentration 750 μg/ml), bovine serum albumin (BSA, final concentration 1 mg/ml) and formyl-methionyl-leucyl-phenylalanine (fMLP, final concentration 10⁻⁷ M) were added for 3 minutes. The cells were chilled on ice, and were spun down. The optical densities in the cell-free supernatant was measured in a spectrophotometer. The median inhibitory concentration (IC₅₀) of the compounds was calculated. The results are shown in Table 1.

TABLE 1
Inhibition of cytokines and PMN-superoxide
production in vitro by compounds of the present invention.

| | The median inhibition concentration (IC₅₀, nM) of | | |
|---|---|---|---|
| | | | PMN- |
| Comp. No. | IL-1β | TNF-α | superoxide |
| Compound 101 | 72 | 17 | 6.3 |
| Ref. comp. | 13 | 7.1 | 5.0 |

Ref. comp.: (4-(2-aminophenylamino)-2-chloro-2′-methylbenzophenone, Compound 156 disclosed in WO 98/32730.

These results show that the compounds of the present invention are able to inhibit the production of IL-1β, TNF-α and PMN-superoxide, and showing a pharmacological activity comparable to a reference compound, thus making them potentially useful in the treatment of inflammatory diseases.

p38α MAP Kinase Assay

Cell Culture

COS-1 cells (derived from African green monkey kidney fibroblast-like cell containing wild-type T antigen under control of the SV40 promotor) were obtained from ATCC (ATCC no. CRL-1650) and grown in growth medium (DMEM without phenolred, 10% FCS, 2 mM L-glutamine, 100U penicillin and 100 μg streptomycin/ml) at 37° C. with 5% CO₂. The cells were passaged twice a week by trypsination (0.25% trypsin, 1 mM EDTA in PBS) and were split 1:10. The medium was changed every second or third day. The cell line was regularly tested with the Mycoplasma PCR Primer Set (Stratagene) and found to be free of Mycoplasma. Tissue culture media, FCS, L-glutamine and penicilin and streptomycin are from Bribco BRL, Gaithersburg, Md., USA.

Transient Expression of COS-1 Cells

On day one COS-1 cells were seeded in 143 cm² petridish with a density of 2×10⁴celler/cm² in growth medium. At day 2 the cells were co-transfected with 5 μg (total) of experimental plasmid DNA, expressing the FLAG-p38α and FLAG-MKK6(EE). The plasmids were introduced into the COS-1 cells in serum-free medium using DOTAP™ (Boehringer-Mannheim, Mannheim, Germany). Plasmid DNA was prepared and purified using the QIAGEN EndoToxin-free Maxiprep-500 kit (Hilden, Germany). Briefly, DNA and DOTAP™ were mixed for exactly 15 min. at 37° C. in the CO₂ incubator. The transfection-mixture was hereafter transferred to a 15-ml falcon-tube and transfection-medium (DMEM with L-Glutamine and Pen./Strep. but without serum) was added to the transfection-mixture, followed by addition to the cell-monolayer. After 4 hours of incubation with DOTAP™ and plasmids, the medium containing double amount of serum was added to the cells bringing the final concentration of serum up to 10%. The cells were then incubated for 24 hours before kinase reaction.

Immunoprecipitation

After 24 hrs of incubation the reaction was stopped by putting the petridish on an ice-bath. The medium was aspirated, and the cell monolayer was washed once in ice-cold PBS (137 mM NaCl, 1.5 mM KH₂PO₄, 2.7 mM KCl, 8.1 mM Na₂HPO₄.2H₂O), and hereafter solubilised for 10 min. in 1.5 ml lysis buffer (50 mM HEPES, pH 7.5, 150 mM NaCl, 10 mM EDTA, 10 mM Na₄P₂O₇, 100 mM NaF, 2 mM Na₃VO₄, 1% Triton-X-100, Pefabloc 500 μM, Leupeptin 10 μg/μl, Aprotinin 10 μg/μl) was added. The cell-monolayer was scraped by a rubber-policeman, and transferred to an Eppendorf tube. The solubilised cells were clarified by centrifugation at 10.000×g for 10 min. at 4° C. The supernatant was transferred to 50 μl prewashed Protein G Sepharose beads in HNT-buffer (30 mM HEPES, pH 7.5, 30 mM NaCl, 0.1% Triton X-100) and were incubated with 2 μg/sample of monoclonal anti-FLAG™ M2 antibody (raised against the FLAG-epitope, NH₂-Asp-Tyr-Lys-Asp-Asp-Asp-Asp-Lys-COOH) for 1 hour at room temperature. The anti-FLAG M2 monoclonal antibody was obtained from Sigma (cat. no. F-3165). Approx. 60 μg protein of clarified cell lysate were added to the preadsorbed anti-FLAG™ antibodies on Protein G Sepharose beads and incubated for 90 min. at 4° C. in a blood sample mixer. After the immunoprecipitation period the Sepharose beads were washed twice in lysis buffer and twice in a kinase reaction buffer (25 mM HEPES pH 7.5, 10 mM magnesium acetate, 50 μM ATP).

Incubation of the compounds with purified p38α kinase

The pre-washed immunoprecipitated anti-FLAG-p38 adsorbed on Protein G Sepharose beads was washed twice in 1×kinase-buffer (25 mM HEPES pH 7.5, 10 mM magnesium acetate, 50 μM ATP), and the supernatant was aspirated. The compounds were diluted in 1×kinase buffer at the appropriate concentration. The compounds were added to the washed immunoprecipitated and activated FLAG-p38 adsorbed on the Protein G Sepharose beads for 30 min. at 30° C. in a volume of 100 μl. Every 10 min. the Eppendorf tubes were tapped to ensure that the beads and the compounds were in the solution. After 30 min. incubation, the beads were spinned down and the supernatant was aspirated.

p38α MAP Kinase Reaction

The kinase reaction was started by adding 1 μg GST-ATF-2 substrate (Santa Cruz, Lajolla, Calif., USA, cat. no. sc-4114) together with 2 μCi γ-³²P-ATP in 1×kinase-buffer per sample. The reaction was allowed to proceed for 30 min. at 30° C., and it was stopped by adding 40 μl of 2×SDS-sample buffer to the kinase reaction. The samples were boiled, spinned down, and resolved on a 15% SDS-PAGE. The dried SDS-PAGE gel was exposed to a Phospho-Imager screen and the radioactive PHAS-1 bands were quantified by the STORM860 Phospho-Imager (Molecular Dynamics, Sunnyvale, Calif., USA) using the ImageQuaNT software.

In this assay, Compound 101 was found to be a potent p38 MAP kinase inhibitor with an IC₅₀ of 93.3 nM.

To study the compounds of the present invention in vivo the 12-O-tetradecanoylphorbol-13-acetate (TPA) induced murine chronic skin inflammation model can be used (De Young, L. M. et al., Agents Actions, 26, 335-341 (1989); Carlson, R. P. et al., Agents Actions, 17, 197-204 (1985); Alford, J. G. et al., Agents Action, 37, (1992); Stanley, P. L. et al., Skin Pharmacol, 4, 262-271 (1991)), cf. description of the method in WO 98/32730 hereby incorporated by reference. These results show that the compounds of the present invention are of the same potency compared to known reference compounds, e.g. hydrocortisone with its known side effects, whereas the compounds of the present invention are well tolerated and are non-toxic. Some members of the present class of compounds show a very low absorption, thus making them especially useful in the treatment of various dermatological diseases.

The invention is described in further detail in the following examples which are not in any way intended to limit the scope of the invention as claimed.

### EXAMPLES

General procedures, preparations and Examples

Specific examples of compounds of formula I are listed in Table 2. All melting points are uncorrected. For ¹H and ¹³C nuclear magnetic resonance (NMR) spectra (300 MHz for ¹H) chemical shift values (δ) (in ppm) are quoted, unless otherwise specified, for deuteriochloroform, tetradeuteriomethanol and hexadeuterodimethyl-sulfoxide solutions relative to internal tetramethylsilane (δ 0.00) or chloroform (¹H NMR δ 7.25, ¹³C NMR δ 76.81). The value for a multiplet (m), eventually defined as doublet (d), triplet (t) or quartet (q), is given at the approximate mid point unless a range is quoted. A signal may also be defined as singlet (s) or broad singlet (bs). The organic solvents used were anhydrous. The term “chromatography” refers to column chromatography using the flash technique and was performed on silica gel.

TABLE 2
Compounds of General formula I

| Comp. No. | | | | | | | | |
|---|---|---|---|---|---|---|---|---|
| Example | | | | | | | | |
| No. | X | HetAr | R13 | R2 | R3 | A | R4 | R5 |
| Comp. 101 Ex. 1 | O | 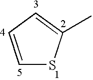 | 3-Cl | 2-Cl | H | 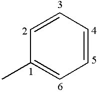 | 2-CH₃ | — |
| Comp. 102 | O | Same as ex.1 | 3-Cl | 2-Cl | H | Same as ex.1 | 2-NO₂ | — |
| Ex. 2 | | | | | | | | |
| Comp 103 | O | Same as ex.1 | 3-Cl | 2-Cl | H | Same as ex.1 | 2-NH₂ | — |
| Ex. 3 | | | | | | | | |
| Comp. 106 | O | Same as ex.1 | 3-CH₃ | 2-Cl | H | Same as ex.1 | 2-CH₃ | — |
| Ex. 6 | | | | | | | | |
| Comp. 107 | O | Same as ex.1 | 3-CH₃ | 2-Cl | H | Same as ex.1 | 2-NO₂ | — |
| Ex. 7 | | | | | | | | |
| Comp. 108 | O | Same as ex.1 | 3-CH₃ | 2-Cl | H | Same as ex.1 | 2-NH₂ | — |
| Ex. 8 | | | | | | | | |
| Comp. 111 Ex. 11 | O | 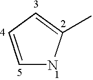 | 1-CH₃ | 2-Cl | H | Same as ex.1 | 2-NO₂ | — |
| Comp. 112 | O | Same as ex.11 | 1-CH₃ | 2-Cl | H | Same as ex.1 | 2-NH₂ | — |
| Ex. 12 | | | | | | | | |
| Comp. 113 | O | Same as ex.11 | 1-CH₃ | 2-Cl | H | Same as ex.1 | 2-CH₃ | — |
| Ex. 13 | | | | | | | | |
| Comp. 114 Ex. 14 | O | 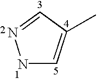 | 1,3,5-tri- CH₃ | 2-Cl | H | Same as ex.1 | 2-CH₃ | — |
| Comp. 115 Ex. 15 | O | 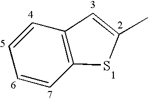 | 3-CH₃ | 2-Cl | H | Same as ex.1 | 2-CH₃ | — |
| Comp. 116 Ex. 16 | O | 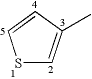 | 4-CH₃ | 2-Cl | H | Same as ex.1 | 3-CH₃ | — |
| Comp. 117 | O | Same as ex.16 | 2,5-di- | 2-Cl | H | Same as ex.1 | 2-CH₃ | — |
| Ex.17 | | | CH₃ | | | | | |
| Comp. 118 Ex. 18 | O | 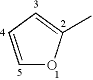 | 3-CH₃ | 2-Cl | H | Same as ex.1 | 2-CH₃ | — |
| Comp. 121 | O | Same as ex.18 | 5-CH₃ | 2-Cl | H | Same as ex.1 | 2-CH₃ | — |
| Ex. 21 | | | | | | | | |
| Comp. 122 Ex. 22 | O | 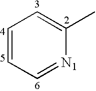 | H | 2-Cl | H | Same as ex.1 | 2-CH₃ | — |
| Comp. 123 | O | Same as ex.22 | 3-Cl | 2-Cl | H | Same as ex.1 | 2-CH₃ | — |
| Ex. 23 | | | | | | | | |
| Comp. 126 Ex. 26 | O | 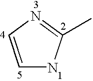 | 1-CH₃ | 2-Cl | H | Same as ex.1 | 2-CH₃ | — |
| Comp. 127 | O | Same as ex.26 | 1-CH₃ | 2-Cl | H | Same as ex.1 | 2-NO₂ | — |
| Ex. 27 | | | | | | | | |
| Comp. 128 | O | Same as ex.26 | 1-CH₃ | 2-Cl | H | same as ex.1 | 2-NH₂ | — |
| Ex. 28 | | | | | | | | |
| Comp. 129 Ex. 29 | O | 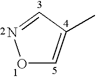 | 3,5-di- CH₃ | 2-Cl | H | Same as ex.1 | 2-CH₃ | |
| Comp. 130 Ex. 30 | O | 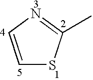 | 4,5-di- CH₃ | 2-Cl | H | Same as ex.1 | 2-CH₃ | — |
| Comp. 131 | O | Same as ex.1 | 5-CH₃ | 2-Cl | H | Same as ex.1 | 2-CH₃ | — |
| Ex. 31 | | | | | | | | |
| Comp. 132 | O | Same as ex.1 | 3-Cl | 2-Cl | H | Same as ex.1 | 2-CN | — |
| Ex. 32 | | | | | | | | |
| Comp. 133 | O | Same as ex.1 | 3-Cl | 2-Cl | H | Same as ex.1 | 2-CF₃ | — |
| Ex. 33 | | | | | | | | |
| Comp. 134 | O | Same as ex.1 | 3-Cl | 2-Cl | H | Same as ex.1 | 2-CH₃ | 4-Br |
| Ex. 34 | | | | | | | | |
| Comp. 135 | O | Same as ex.1 | 3-Cl | 2-Cl | H | Same as ex.1 | 2-CN | 4-Br |
| Ex. 35 | | | | | | | | |
| Comp. 136 Ex. 36 | O | Same as ex.1 | 3-Cl | 2-Cl | H | 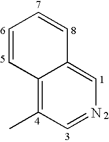 | H | H |
| Comp. 137 | O | Same as ex.1 | 3-Cl | 2-Cl | H | Same as ex.1 | 2,3,5,6- | 4-Br |
| Ex. 37 | | | | | | | tetra-CH₃ | |
| Comp. 138 | O | Same as ex.1 | 3-Cl | 2-Cl | H | Same as ex.1 | 2-Cl | 4-Br |
| Ex. 38 | | | | | | | | |
| Comp. 139 | O | Same as ex.1 | 3-Cl | 2-Cl | H | Same as ex.1 | 2-NO₂ | 4-Br |
| Ex. 39 | | | | | | | | |
| Comp. 140 | O | Same as ex.1 | 3-Cl | 2-Cl | H | Same as ex.1 | 2-NH₂ | 4-Br |
| Ex. 40 | | | | | | | | |
| Comp. 141 | O | Same as ex.1 | 3-Cl | 2-Cl | H | Same as ex.1 | — | — |
| Ex. 41 | | | | | | | | |
| Comp. 142 | O | Same as ex.1 | 3-Cl | 2-Cl | H | Same as ex.1 | 2-CF₃ | 4-Br |
| Ex. 42 | | | | | | | | |
| Comp. 143 Ex. 43 | O | Same as ex.1 | 3-Cl | 2-Cl | H | 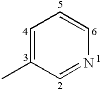 | — | — |
| Comp. 146 Ex. 46 | O | 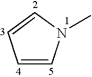 | — | 2-Cl | H | Same as ex.1 | 2-CH₃ | — |
| Comp. 149 | O | Same as ex. 11 | 3,5- | 2-Cl | H | Same as ex.1 | 2-CH₃ | — |
| Ex. 49 | | | diCH₃ | | | | | |
| Comp. 150 | O | Same as ex. 1 | 3-Cl | 2-Cl | H | Same as ex.1 | 2-NH₂ | 4-CF₃ |
| Ex. 50 | | | | | | | | |
| Comp. 151 Ex. 51 | O | 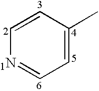 | 3-Cl | 2-Cl | H | Same as ex.1 | 2-CH₃ | — |
| Comp. 152 | O | Same as ex. 15 | 3-CH₃-5- | 2-Cl | H | Same as ex.1 | 2-CH₃ | — |
| Ex. 52 | | | Cl | | | | | |
| Comp. 153 Ex. 53 | O | 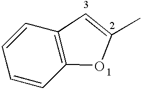 | — | 2-Cl | H | Same as ex.1 | 2-CH₃ | — |
| Comp. 154 Ex. 54 | O | 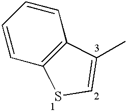 | — | 2-Cl | H | Same as ex.1 | 2-CH₃ | — |
| Comp. 155 | O | Same as ex.15 | 3,5-di | 2-Cl | H | Same as ex.1 | 2-CH₃ | — |
| Ex.55 | | | CH₃ | | | | | |
| Comp. 156 Ex. 56 | O | 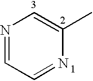 | 3-Cl | 2-Cl | H | Same as ex.1 | 2-CH₃ | — |
| Comp. 159 Ex. 59 | O | 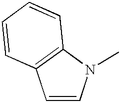 | — | 2-Cl | H | Same as ex.1 | 2-CH₃ | — |
| Comp. 160 | O | Same as ex.16 | 2,5-diCl | 2-Cl | H | Same as ex.1 | 2-CH₃ | — |
| Ex. 60 | | | | | | | | |
| Comp.161 Ex. 61 | O | 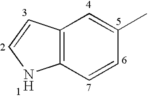 | — | 2-Cl | H | Same as ex.1 | 2-CH₃ | — |
| Comp. 162 Ex. 62 | O | 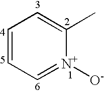 | 3-Cl | 2-Cl | H | Same as ex.1 | 2-CH₃ | — |
| Comp. 163 | O | Same as ex.1 | 3-Cl | 2-Cl | CH₃ | Same as ex.1 | 2-CH₃ | — |
| Ex. 63 | | | | | | | | |

The numbering in Table 2 refers to the numbering in the formula below, R₁₃ is as defined above in Scheme 1 and R₂, R₃ and A are defined as for general formula I_{b}:

General Procedure 1

Coupling of compounds of the general formula II with compounds of the general formula VIII to give compounds of the general formula I, or a protected derivative thereof.

The heteroarene (1.0 eq.) with the general formula II was dissolved in dry THF or dry Et₂O and cooled with stirring under an atmosphere of argon. The organometallic base (n-butyllithium, 1.6 M solution in hexane or i-propylmagnesiumchloride, 2 M solution in Et₂O) was then added dropwise over 2 min. The resulting mixture was stirred for the metallation time before being added to a cold (same temperature as metallation temperature) THF/Et₂O solution of compound with the general formula VIII (0.5 eq.). The reaction mixture was allowed to warm to 20° C. over 2-3 h. The resulting solution was quenched with saturated aqueous NH₄Cl. The aqueous phase was extracted with EtOAc (3 times). The organic phases were combined, dried (MgSO₄), filtered, and concentrated in vacuo to afford the crude product. Further purification was done by flash chromatography and/or crystallization to give the title compound of formula I, or a protected derivative thereof.

Similarly: Coupling of compounds of the general formula II with compounds of the general formula III (1 eq.) to give compounds of the general formula IV, or a protected derivative thereof.

General Procedure 2

Coupling of compounds of the general formula V with compounds of the general formula VI to give compounds of the general formula I, or a protected derivative thereof.

An amine of the general formula V (1 eq.) was dissolved in DMSO (0.1-0.2 M), and a (hetero)arylchloride or (hetero)arylfluoride (1 eq.) of the general formula VI was added. Potassium t-butoxide, 2 eq., or 3 eq. if compound VI was provided as the hydrochloride, was dissolved in DMSO (0.2-0.4M), and the solution was added dropwise to the reaction mixture. The reaction was stirred under an argon atmosphere at rt for 12-48 h. The reaction mixture was poured into water and extracted with EtOAc three times. The combined organic phases were washed with saturated aq. NaCl, dried (MgSO₄), filtered and concentrated in vacuo, and the crude product was purified by flash chromatography to afford the coupled product with the general formula I, or a protected derivative thereof.

General Procedure 3

Coupling of compounds of the general formula VI with compounds of the general formula V to give compounds of the general formula I, or a protected derivative thereof.

In a screw cap vessel an amine (1.0 eq.) with the general formula V was dissolved in 1,4-dioxane or toluene, and a halogenide (1.0-1.1 eq.), with the general formula VI was added. The vessel was flushed with argon, the base (Cs₂CO₃ or NaOt—Bu, 1.4 eq.), Pd₂(dba)₃ (0.02 eq.), and BINAP (0.04 eq.) were added, the vessel was again flushed with argon and closed. The resulting suspension was first shaken vigorously at room temperature for 5 min and then at 100-110° C. for 4-20 h or until the halogenide VI had disappeared as seen on TLC. A second portion of palladium source was eventually added after 4 h. The reaction mixture was allowed to cool to room temperature. The reaction mixture was poured into a mixture of EtOAc and water. The organic phase was separated, and the water phase was extracted with EtOAc three times. The combined organic phases were washed with water and saturated aqueous NaCl, dried (MgSO₄), filtered and concentrated in vacuo. The residue was purified either by chromatography and/or crystallization to afford the coupled product with the general formula I, or a protected derivative thereof.

The same procedure may be followed with an amine of general formula VII and a halogenide of general formula VI giving access to compounds of general formula VIII.

General Procedure 4

Reduction of compounds of the general formula IV with stannous chloride dehydrate to give compounds of the general formula V, or a protected derivative thereof.

A mixture of a compound with the general formula IV (5 mmol) and stannous chloride dihydrate (5.64 g, 25 mmol) in absolute ethanol (50 ml) was heated to 70° C. under argon. After 1 hour, or until the starting material had disappeared as seen on TLC, the solution was allowed to cool to room temperature and then poured into ice/water. The pH was made alkaline by the addition of saturated sodium hydroxide (50 ml) before being extracted with ethyl acetate (3×100 ml). The organic phase was dried (MgSO₄), filtered and evaporated to afford the crude product. The crude product was further purified either by crystallization or flash chromatography to yield the title compound or a protected derivative thereof.

Similarly: Reduction of compounds of the general formula IIIa to give compounds of the general formula VII, or a protected derivative thereof.

Similarly: Reduction of compounds of the general formula I to give compounds of the general formula I, or a protected derivative thereof.

General Procedure 5

Deprotection of silylated derivatives of to give compounds of general formula I.

The protected derivative is dissolved in THF. Addition of tetrabutylammonium fluoride, trihydrate (1 eq.). Stirring at rt for 1 h. Addition of 0.5% aq. NaHCO3 until pH=8. Extraction with ethyl acetate. The organic phase was dried (MgSO₄), filtered and evaporated to afford the crude product. The crude product was further purified either by crystallization or flash chromatography to yield the title compound.

Preparation 1:

2-Chloro-4-nitrophenyl 3-chloro-2-thienyl ketone, (Compound 1)

General procedure: 1

Starting compound II: 2-Bromo-3-chlorothiophene (3.95 g, 20 mmol)

Starting compound III: 2-Chloro-4-nitro-benzoyl chloride (22 mmol) in THF (10 mL)

Solvent: THF (30 mL)

Base: i-PrMgCl (22 mmol)

Metallation temperature: −35° C.

Metallation time: 30 min

Purification: Chromatography using EtOAc/pentane 1:10 as eluant

Product: compound 1, 2.7 g oil (44%)

¹³C NMR (CDCl₃): δ 184.3, 149.1, 144.3, 135.2, 134.7, 132.4, 131.9, 131.0, 129.1, 125.2, 122.2

Preparation 2:

4-Amino-2-chlorophenyl 3-chloro-2-thienyl ketone, (Compound 2)

General procedure: 4

Starting compound IV: Compound 1

Purification: used without further purification (95%)

¹³C NMR (CDCl₃): δ 185.8, 150.0, 136.7, 133.8, 131.8, 131.6, 130.2, 129.8, 127.8, 115.6, 112.5

Preparation 3:

N-methoxy-N-methyl-2-chloro-4-nitrobenzamide. (Compound 3)

2-Chloro-4-nitrobenzoyl chloride (1 eq.) was dissolved in dry CH₂Cl₂ under argon and N-methoxy-N-methylamine. hydrochloride (1.1 eq.) was added. The reaction mixture was cooled to 0° C. and dry pyridine (2.2 eq.) was added dropwise. Stirring at 0° C. for 30 min. and at rt for 4 h. Addition of Et₂O. The organic phase was washed with water (3 times), dried (MgSO₄), filtered, and concentrated in vacuo. Purification by crystallization (EtOAc) (99%)

¹³C NMR (CDCl₃): δ 166.4, 148.5, 141.5, 132.2, 128.4, 124.8, 121.7, 61.6, 32.3

Preparation 4:

N¹-methoxy-N¹-methyl-4-amino-2-chlorobenzamide. (Compound 4)

General procedure: 4

Starting compound IV: compound 3

Purification: Chromatography using EtOAc-PE 2:1

Product: Compound 4, oil (90%)

¹³C NMR (CDCl₃): δ 148.5, 131.9, 129.2, 124.2, 115.1, 112.9, 61.0, 33.5

Preparation 5:

N¹-methoxy-N¹-methyl-4-(2-tolylamino)-2-chlorobenzamide. (Compound 5)

General procedure: 3

Starting compound VI: 2-bromotoluene (0.38 g, 2.2 mmol)

Starting compound V: compound 4 (0.43 g, 2.0 mmol)

Solvent: 1,4-dioxan (10 mL)

Base: Cs₂CO₃ (0.46 g, 4.8 mmol)

Reaction time: 21 h

Reaction temperature: 100° C.

Purification: Chromatography using CH₂Cl₂-EtOAc: 10:1

Product: Compound 5, 0.30 g oil (50%)

¹³C NMR (CDCl₃): δ 171.1, 147.1, 139.1, 132.0, 131.4, 131.3, 129.1, 127.0, 125.2, 124.4, 122.5, 115.5, 113.4, 61.1, 33.6, 17.9

Protection: Compound 5 was dissolved in dry THF under argon and cooled to −35° C. Lithium bis (trimethylsilyl)amide (1M in hexane, 1.2 eq.) was added dropwise. Stirring for 30 min before addition of trialkylsilylchloride (1.2 eq.). The resulting mixture was allowed to warm up to rt over 1 h under stirring. Addition of water, the aqueous phase was extracted with EtOAc (3 times). The combined extracts were dried (MgSO₄), filtered and evaporated to afford the crude product. The crude product was further purified by flash chromatography.

Compound 6

Protecting group: trimethylsilyl

Purification: Chromatography using PE-EtOAc: 2:1

Product: Compound 6, crystals (71%) mp 91-94° C.

¹³C NMR (CDCl₃): δ 151.2, 142.9, 137.3, 131.4, 131.2, 130.2, 128.3, 127.3, 126.8, 123.4, 116.2, 113.9, 60.8, 33.9, 17.7, 0.6

Compound 7

Protecting group: triisopropylsilyl

Purification: Chromatography using PE-EtOAc: 3:1

Product: Compound 7, crystals (94%) mp 94-97° C.

¹H NMR (CDCl₃): δ 7.20 (4H,m), 7.06 (1H,d), 6.58 (1H,d), 6.50 (1H,d), 3.56 (3H,s,Br), 3.26 (3H,s), 2.13 (3H,s), 1.40 (3H,m), 1.05 (18H,d)

### Example 1

4-(2-tolylamino)-2-chlorophenyl 3-chloro-2-thienyl ketone (Compound 101)

General procedure: 3

Starting compound VI: 2-bromotoluene (0.38 g, 2.2 mmol)

Starting compound V: compound 2 (0.54 g, 2.0 mmol)

Solvent: 1,4-dioxan (10 mL)

Base: t-BuONa (0. g, 4.8 mmol)

Reaction time: 16 h

Reaction temperature: 100° C.

Purification: Chromatography using PE-EtOAc 15:1

Product: Compound 101, 0.22 g crystals (31%) mp 121-123° C.

¹³C NMR (CDCl₃): δ 185.7, 148.7, 138.3, 136.7, 133.9, 132.1, 131.7, 131.6, 131.4, 130.3, 129.8, 128.4, 127.1, 125.2, 123.5, 115.5, 112.7, 17.9

### Example 2

4-(2-nitrophenylamino)-2-chlorophenyl 3-chloro-2-thienyl ketone (Compound 102)

General procedure: 2

Starting compound VI: 2-fluoro-nitrobenzene (0.23 mL, 2.2 mmol)

Starting compound V: compound 2 (0.54 g, 2.0 mmol)

Solvent: DMSO (10 mL)

Base: t-BuOK (0.45 g, 4.0 mmol) in DMSO (10 mL)

Reaction time: 5 days

Reaction temperature: rt

Purification: Chromatography using EtOAc:PE 1:10

Product: Compound 102, 0.40 g oil (51%)

¹³C NMR (CDCl₃): δ 185.5, 142.5, 140.4, 135.8, 135.0, 134.3, 133.0, 130.9, 130.7, 130.5, 126.9, 123.0, 120.3, 119.6, 117.0

### Example 3

4-(2-aminophenylamino)-2-chlorophenyl 3-chloro-2-thienyl ketone (Compound 103)

General procedure: 4

Starting compound I: compound 102 (0.79 g, 2.0 mmol)

Purification: Chromatography using EtOAc:petroleum ether 1:8

Product: Compound 103, 0.51 g crystals (70%)

¹³C NMR (CDCl₃): δ 185.7, 149.2, 142.8, 136.7, 134.0, 131.7, 131.6, 130.3, 129.8, 128.2, 127.6, 126.8, 125.5, 119.2, 116.4, 115.0, 112.1

### Example 4

2-chloro-4-nitrophenyl 3-methyl-2-thienyl ketone (Compound 104)

General procedure: 1

Starting compound II: 2-bromo-3-methylthiophene (0.35 g, 2.0 mmol)

Starting compound III: 2-chloro-4-nitrobenzoyl chloride (0.48 g, 2.2 mmol) in THF (1 mL)

Solvent: THF (3 mL)

Base: i-PrMgCl (2.2 mmol)

Metallation temperature: −35° C.

Metallation time: 30 min

Purification: Chromatography using pentane-EtOAc 20:1

Product: Compound 104, 0.16 g oil (28%)

¹³C NMR (CDCl₃): δ 185.4, 148.8, 148.0, 145.6, 135.1, 134.0, 133.2, 132.3, 128.9, 125.4, 121.9, 16.7

### Example 5

4-amino-2-chlorophenyl 3-methyl-3-thienyl ketone (Compound 105)

General procedure: 4

Starting compound IV: compound 104 (0.56 g, 2.0 mmol)

Reaction time: 2 h

Product: Compound 105, 0.39 g oil (78%).

¹³C NMR (CDCl₃): δ 187.7, 149.3, 145.5, 137.3, 133.0, 132.4, 131.7, 130.9, 129.7, 115.7, 112.4, 16.3

### Example 6

4-(2-tolylamino)-2-chlorophenyl 3-methyl-2-thienyl ketone (Compound 106)

General procedure: 3

Starting compound VI: 2-bromotoluene (0.38 g, 2.2 mmol)

Starting compound V: compound 105 (0.50 g, 2.0 mmol)

Solvent: 1,4-dioxane (10 mL)

Base: Cs₂CO₃ (0.91 g, 2.8 mmol)

Reaction time: 24 h

Reaction temperature: 100° C.

Purification: Chromatography using EtOAc-PE 1:7

Product: Compound 106, 0.33 g oil (48%)

¹³C NMR (CDCl₃): δ 187.6, 148.0, 145.6, 138.6, 137.2, 133.1, 132.4, 131.8, 131.8, 131.3, 130.8, 130.1, 127.0, 124.9, 123.1, 115.7, 112.6, 17.9, 16.4

### Example 7

4-(2-nitrophenylamino)-2-chlorophenyl 3-methyl-2-thienyl ketone (Compound 107)

General procedure: 2

Starting compound VI: 2-fluoro-nitrobenzene (0.23 mL, 2.2 mmol)

Starting compound V: compound 105 (0.50 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 1:6

Product: Compound 107, 0.54 oil (72%)

¹H NMR (CDCl₃): δ 9.43 (1H,s), 8.22 (1H,dd), 7.55 (1H,d), 7.5-7.4 (3H,m), 7.37 (1H,d), 7.23 (1H,dd), 7.00 (1H,d), 6.93 (1H,m), 2.47 (3H,s)

### Example 8

4-(2-aminophenylamino)-2-chlorophenyl 3-methyl-2-thienyl ketone (Compound 108)

General procedure: 4

Starting compound I: compound 107 (0.74 g, 2.0 mmol)

Reaction time: 5 h

Product: Compound 105, 0.28 g oil (14%).

¹H NMR (CDCl₃): δ 7.48 (1H,d), 7.32 (1H,d), 7.18-7.04 (2H,m), 6.95 (1H,d), 6.85-6.75 (2H,m), 6.72 (1H,d), 6.60 (1H,dd), 5.98 (1H,Br), 4.2 (2H,Br), 2.43 (3H,s)

### Example 9

2-chloro-4-nitrophenyl 1-methyl-2-pyrrolyl ketone (Compound 109)

1-Methylpyrrole (0.32 g, 4 mmol) and N,N,N′,N′-tetramethylethylenediamine (0.51 mg, 4.4 mmol) were dissolved in dry Et₂O (3 mL) and cooled with stirring to −78° C. under an atmosphere of argon. n-Butyllithium (1.1 eq., 1.6 M solution in hexane) was then added dropwise over 2 min. The resulting mixture was allowed to warm up to rt over 30 min and was then heated to reflux for 1h. Cooling to −78° C. before addition of a cold (−78° C.) ethereal solution of 2-chloro-4-nitrobenzoyl chloride (0.97 g, 4.4 mmol in 5 mL Et₂O). The reaction mixture was allowed to warm to 20° C. over 3 h. The resulting solution was quenched with saturated aqueous NH₄Cl. The aqueous phase was extracted with EtOAc (3 times). The organic phases were combined, dried (MgSO₄), filtered, and concentrated in vacuo to afford the crude product. Flash chromatography with EtOAc-PE 1:8 then 1:5 as eluant afforded compound 109, 0.16 g oil (15%).

¹H NMR (CDCl₃): δ 8.32 (1H,d), 8.18 (1H,dd), 7.57 (1H,d), 7.01 (1H,dd), 6.43 (1H,dd), 6.15 (1H,dd), 4.10 (3H,s)

### Example 10

4-amino-2-chlorophenyl 1-methyl-2-pyrrolyl ketone (Compound 110)

General procedure: 4

Starting compound IV: compound 109 (0.51 g, 2.0 mmol)

Reaction time: 1 h

Purification: Chromatography using EtOAc-PE 1:1

Product: Compound 110, 0.20 g oil (43%).

¹³C NMR (CDCl₃): δ 185.3, 148.7, 133.2, 131.7, 131.4, 131.2, 129.3, 123.1, 115.8, 112.0, 108.2, 37.4

### Example 11

4-(2-nitrophenylamino)-2-chlorophenyl 1-methyl-2-pyrrolyl ketone (Compound 111)

General procedure: 2

Starting compound VI: 2-fluoro-nitrobenzene (0.23 mL, 2.2 mmol)

Starting compound V: compound 110 (0.47 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 1:3

Product: Compound 107, 0.64 oil (93%)

¹³C NMR (CDCl₃): δ 183.5, 141.2, 141.0, 135.8, 134.6, 132.9, 132.6, 130.6, 130.5, 126.8, 123.9, 123.8, 120.2, 119.1, 116.7, 108.7, 37.6

### Example 12

4-(2-aminophenylamino)-2-chlorophenyl 1-methyl-2-pyrrolyl ketone (Compound 112)

General procedure: 4

Starting compound I: Compound 111 (0.34 g, 1.0 mmol)

Reaction time: 1 h

Product: Compound 112, 0.10 g oil (32%)

¹H NMR (CDCl₃): δ 7.30 (1H,d), 7.11 (2H,m), 6.89 (1H,s), 6.80 (2H,m), 6.73 (1H,d), 6.57 (2H,m), 6.10 (1H,dd), 5.39 (1H,Br), 4.05 (3H,s), 3.8 (2H,Br)

### Example 13

4-(2-tolylamino)-2-chlorophenyl 1-methyl-2-pyrrolyl ketone (Compound 113)

General procedure: 3

Starting compound VI: 2-bromotoluene (0.38 g, 2.2 mmol)

Starting compound V: compound 110 (0.47 g, 2.0 mmol)

Solvent: 1,4-dioxane (10 mL)

Base: Cs₂CO₃ (0.91 g, 2.8 mmol)

Reaction time: 24 h

Reaction temperature: 100° C.

Purification: Chromatography using EtOAc-PE 1:10

Product: Compound 113, 0.49 g oil (76%)

¹³C NMR (CDCl₃): δ 184.2, 147.3, 138.9, 133.3, 131.8, 131.5, 131.3, 131.2, 131.2, 130.0, 127.0, 124.5, 123.2, 122.7, 116.1, 112.4, 108.2, 37.4, 17.9

### Example 14

4-(2-tolylamino)-2-chlorophenyl 1,3,5-trimethyl-4-pyrazolyl ketone (Compound 114)

General procedure: 1

Starting compound II: 1,3,5-trimethyl-4-bromopyrazole (0.38 g, 2.0 mmol)

Starting compound VIII: compound 6 (0.38 g, 1.0 mmol) in THF (6 mL)

Solvent: THF (4 mL)

Base: n-BuLi (2.1 mmol)

Metallation temperature: −78° C.

Metallation time: 15 min

Purification by chromatography using EtOAc-PE 1:2 then 1:1

Product: Compound 114, 0.22 g (63%) oil, the silyl group was lost upon work-up.

¹³C NMR (CDCl₃): δ 190.0, 149.7, 148.0, 143.7, 138.8, 132.7, 131.9, 131.3, 130.7, 130.7, 127.0, 124.8, 123.1, 119.3, 115.7, 113.1, 35.8, 17.9, 13.7, 11.1

### Example 15

3-methyl-2-benzothienyl 4-(2-tolylamino)-2-chlorophenyl ketone (Compound 115)

General procedure: 1

Starting compound II: 3-methylbenzothiophene (0.75 g, 2.0 mmol)

Starting compound VIII: compound 6 (0.38 g, 1.0 mmol) in THF (6 mL)

Solvent: THF (4 mL)

Base: n-BuLi (2.1 mmol)

Metallation temperature: −78° C.

Metallation time: 10 min

Purification: Chromatography using EtOAc-PE 1:5 followed by RP-HPLC

Product: Compound 115, 0.02 g (6%) oil, the silyl group was lost upon work-up.

¹H NMR (CDCl₃): δ 7.84 (2H,m), 7.45 (2H,m), 7.39 (1H,d), 7.28 (2H,m), 7.23 (1H,m), 7.11 (1H,m), 6.85 1H,d), 6.72 (1H,dd), 5.6 (1H,Br), 2.56 (3H,s), 2.27 (3H,s)

### Example 16

4-(2-tolylamino)-2-chlorophenyl 4-methyl-3-thienyl ketone (Compound 116)

General procedure: 1

Starting compound VI: 3-Bromo-4-methylthiophene, (0.35 g, 2.0 mmol)

Starting compound VIII: compound 6 (0.38 g, 1.0 mmol)

Solvent: THF

Base: n-BuLi (2.1 mmol)

Metallation temperature: −78° C.

Metallation time: 5 min

Purification: Chromatography using EtOAc:petroleum ether 1:10 followed by RP-HPLC

Product: Compound 116, 0.04 g (6%) crystals, the silyl group was lost upon work-up.

¹H NMR (CDCl₃): δ 7.61 (1H,d), 7.30 (1H,d), 7.25 (2H,m), 7.20 (1H,m), 7.09 (1H,m), 6.94 (1H,m), 6.82 (1H,d), 6.67 (1H,dd), 5.75 (1H,Br), 2.47 (3H,d, J=1.14 Hz), 2.25 (3H,s)

### Example 17

4-(2-tolylamino)-2-chlorophenyl 2,5-dimethyl-3-thienyl ketone (Compound 117)

2,5-Dimethylthiophene (10 mmol) was stirred in a 1:6 mixture of H₂SO₄/H₂O at rt. N-Bromosuccinimide (1.1 eq.) was added, stirring for 2 h. Extraction with CH₂Cl₂ (3 times), the combined extracts were washed with aqueous saturated NaHCO₃, dried over MgSO₄, filtered and concentrated in vacuo. Kugelrohr distillation affords 3-bromo-2,5-dimethylthiophene.

¹³C NMR (CDCl₃): δ 136.9, 131.6, 127.6, 108.0, 15.3, 14.5

General procedure: 1

Starting compound II: 3-Bromo-2,5-dimethylthiophene, (0.38 g, 2.0 mmol)

Starting compound VIII: compound 6 (0.38 g, 1.0 mmol)

Solvent: THF (4+6 mL)

Base: n-BuLi (2.1 mmol)

Metallation temperature: −78° C.

Metallation time: 10 min

Purification: Chromatography using EtOAc:petroleum ether 1:20, followed by RP-HPLC

Product: Compound 117

¹H NMR (CDCl₃): δ 7.31-7.17 (4H,m), 7.10 (1H,td), 6.82 (1H,d), 6.72 (1H,m), 6.69 (1H,dd), 2.57 (3H,s), 2.37 (3H,s), 2.26 (3H,s)

### Example 18

3-methyl-2-furyl 4-(2-tolylamino)-2-chlorophenyl ketone (Compound 118)

2-Bromo-3-methylfuran:

N-Bromosuccinimide (1 eq.) and azoisobutyronitrile (0.05 eq.) were supended in anhydrous diethylether under argon. 3-Methylfuran (2 mmol) was added and the suspension was heated to 33° C. for 3 h. The solid was filtered off. The ether-phase was washed with 1% aqueous NaHCO₃, dried over MgSO₄ containing CaCO₃ (6 mg) and hydroquinone (1.5 mg). Filtration into a flask containing CaCO₃ (6 mg) and hydroquinone (1.5 mg). Careful evaporation of the solvent.

¹H NMR (CCl₄): δ 7.30 (1H,d), 6.20 (1H,d), 1.98 (3H,s)

General procedure: 1

Starting compound II: 2-Bromo-3-methylfuran, (0.32 g, 2.0 mmol)

Starting compound VIII: compound 6 (0.38 g, 1.0 mmol) in THF (6 mL)

Solvent: THF (4 mL)

Base: n-BuLi (2.1 mmol)

Metallation temperature: −78° C.

Metallation time: 45 min

Purification: Chromatography using EtOAc-PE 1:8

Product: Compound 118, 0.26 g (81%).

¹H NMR (CDCl₃): δ 7.44 (1H,d), 7.36 (1H,d), 7.3-7.15 (3H,m), 7.10 (1H,m), 6.83 (1H,d), 6.72 (1H,dd), 6.42 (1H,d), 5.67 (1H,s), 2.36 (3H,s), 2.25 (3H,s)

### Example 19

5-methyl-2-furyl 2-chloro-4-nitrophenyl ketone (Compound 119)

General procedure: 1

Starting compound II: 2-methylfuran (0.18 mL, 2.0 mmol)

Starting compound III: 2-chloro-4-nitrobenzoyl chloride (0.48 g, 2.2 mmol) in THF (2 mL) at −78° C.

Solvent: THF (2 mL)

Base: n-BuLi (2.2 mmol)

Metallation temperature: −5° C.

Metallation time: 30 min

Purification: Chromatography using pentane-EtOAc 20:1

Product: Compound 119, 0.08 g crystals (16%)

¹³C NMR (CDCl₃): δ 179.0, 160.9, 150.1, 149.0, 143.4, 133.1, 129.8, 125.4, 124.4, 121.7, 110.1, 14.3

### Example 20

5-methyl-2-furyl 4-amino-2-chlorophenyl ketone (Compound 120)

General procedure: 4

Starting compound IV: compound 119 (0.53 g, 2.0 mmol)

Reaction time: 1 h

Purification: Chromatography using EtOAc-PE 1:1

Product: Compound 120, 0.23 g oil (48%).

¹³C NMR (CDCl₃): δ 181.0, 158.9, 151.4, 149.5, 133.7, 131.6, 127.0, 123.0, 115.9, 112.1, 109.1, 14.2

### Example 21

5-methyl-2-furyl 4-(2-tolylamino)-2-chlorophenyl ketone (Compound 121)

General procedure: 3

Starting compound VI: 2-bromotoluene (0.38 g, 2.2 mmol)

Starting compound V: compound 120 (0.47 g, 2.0 mmol)

Solvent: 1,4-dioxane (10 mL)

Base: Cs₂CO₃ (0.91 g, 2.8 mmol)

Reaction time: 44 h

Reaction temperature: 100° C.

Purification: Chromatography using CH₂Cl₂

Product: Compound 121, 0.09 g oil (14%)

¹³C NMR (CDCl₃): δ 180.8, 159.0, 151.4, 148.1, 138.5, 133.9, 131.9, 131.5, 131.4, 127.8, 127.1, 125.0, 123.3, 123.0, 116.0, 112.3, 109.1, 17.9, 14.2

### Example 22

4-(2-tolylamino)-2-chlorophenyl 2-pyridyl ketone (Compound 122)

General procedure: 1

Starting compound II: 2-bromopyridine, (0.32 g, 2.0 mmol)

Starting compound VIII: compound 7 (0.46 g, 1.0 mmol)

Solvent: THF (3+2 mL)

Base: n-BuLi (4.0 mmol)

Metallation temperature: −78° C.

Metallation time: 5 min

Purification: Chromatography using EtOAc-PE 1:6 affords 0.24 g of protected derivative

¹H NMR (CDCl₃): δ 8.65 (1H,m), 7.95 (1H,dt), 7.83 (1H,td), 7.41 (1H,d), 7.40 (1H,m), 7.23 (3H,m), 7.15 (1H,m), 6.57 (1H,d), 6.46 (1H,dd), 2.18 (3H,s), 1.43 (3H,m), 1.07 (18H,d)

Deprotection following general procedure 5

Purification: Chromatography using EtOAc-PE 1:3

Product: Compound 122, 0.15 g (24%) crystals

¹H NMR (CDCl₃): δ 8.67 (1H,m), 8.02 (1H,m), 7.86 (1H,dt), 7.50 (1H,d), 7.44 (1H,m), 7.25 (2H,m), 7.21 (1H,m), 7.10 (1H,dt), 6.81 (1H,d), 6.72 (1H,dd), 5.79 (1H,s,Br), 2.25 (3H,s)

### Example 23

4-(2-tolylamino)-2-chlorophenyl 3-chloro-2-pyridyl ketone (Compound 123)

General procedure: 1

Starting compound II: 3-chloropyridine, (0.19 mL, 2.0 mmol)

Starting compound VIII: compound 7 (0.46 g, 1.0 mmol) in Et₂O (2 mL)

Solvent: Et₂O (4 mL)

Base: n-BuLi (2.0 mmol)+1,4-diazabicyclo[2.2.2]octane (2.0 mmol)

Metallation temperature: −60° C.

Metallation time: 2 h

Purification: Chromatography using EtOAc-PE 1:4 affords 0.23 g (22%) of protected derivative

¹H NMR (CDCl₃): δ 8.48 (1H,dd), 7.76 (1H,dd), 7.56 (1H,d), 7.30 (1H,dd), 7.22 (3H,m), 7.13 (1H,m), 6.49 (1H,m), 6.48 (1H,dd), 2.16 (3H,s), 1.42 (3H,m), 1.06 (18H,d)

Deprotection following general procedure 5

Purification: Chromatography using EtOAc-PE 1:3

Product: Compound 123, 0.15 g (99%) crystals

¹H NMR (CDCl₃): δ 8.50 (dd,1H), 7.79 (dd,1H), 7.69 (d,1H), 7.32 (dd,1H), 7.25 (m,4H), 7.15 (m,1H), 6.71 (d,1H), 6.67 (dd,1H), 5.89 (s,Br,2H), 2.24 (s,3H)

### Example 24

1-methyl-2-imidazolyl 2-chloro-4-nitrophenyl ketone (Compound 124)

General procedure: 1

Starting compound II: 1-methylimidazol (0.16 mL, 2.0 mmol)

Starting compound III: 2-chloro-4-nitrobenzoyl chloride (0.48 g, 2.2 mmol) in THF (2 mL)

Solvent: THF (2 mL)

Base: n-BuLi (2.2 mmol)

Metallation temperature: −78° C.

Metallation time: 30 min

Purification: Chromatography using CH₂Cl₂-EtOAc 9:1

Product: Compound 124, 0.18 g crystals (34%)

¹³C NMR (CDCl₃): δ 183.0, 148.8, 143.9, 142.2, 132.9, 131.2, 130.2, 128.3, 125.3, 121.5, 36.3

### Example 25

1-methyl-2-imidazolyl 4-amino-2-chlorophenyl ketone (Compound 125)

General procedure: 4

Starting compound IV: compound 124 (0.53 g, 2.0 mmol)

Reaction time: 1 h

Purification: Chromatography using EtOAc-CH₂Cl₂ 4:1

Product: Compound 125, 0.36 g oil (76%).

¹³C NMR (CDCl₃): δ 183.8, 150.1, 143.7, 134.7, 133.7, 129.4, 126.7, 126.6, 116.0, 111.9, 36.1

### Example 26

1-methyl-2-imidazolyl 4-(2-tolylamino)-2-chlorophenyl ketone (Compound 126)

General procedure: 3

Starting compound VI: 2-bromotoluene (0.038 g, 0.2 mmol)

Starting compound V: compound 125 (0.047 g, 0.2 mmol)

Solvent: 1,4-dioxane (1 mL)

Base: Cs₂CO₃ (0.09 g, 0.28 mmol)

Reaction time: 44 h (2 portions of palladium-source)

Reaction temperature: 100° C.

Purification: Chromatography using CH₂Cl₂-EtOAc 9:1 followed by RP-HPLC

Product: Compound 126, 8 mg oil (11%)

¹³C NMR (CDCl₃): δ 183.7, 148.7, 143.7, 138.4, 134.8, 133.6, 132.1, 131.3, 129.5, 127.3, 127.0, 126.7, 125.1, 123.7, 115.9, 112.0, 36.2, 17.9

### Example 27

1-methyl-2-imidazolyl 4-(2-nitrophenyl)amino-2-chlorophenyl ketone (Compound 127)

General procedure: 2

Starting compound VI: 2-fluoro-nitrobenzene (0.23 mL, 2.2 mmol)

Starting compound V: compound 125 (0.47 g, 2.0 mmol)

Solvent: DMSO (10 mL)

Base: t-BuOK (0.45 g, 2 mmol)

Reaction time: 4 days

Reaction temperature: rt

Purification: Chromatography using EtOAc-PE 1:1 then EtOAc-PE-NEt₃ 1:1:1%

Product: Compound 127, 0.35 oil (49%)

¹³C NMR (CDCl₃): δ 183.8, 143.1, 142.3, 140.4, 135.7, 134.9, 133.8, 133.5, 132.2, 130.3, 127.5, 126.8, 123.3, 119.5, 119.5, 117.2, 36.3

### Example 28

1-methyl-2-imidazolyl 4-(2-aminophenylamino)-2-chlorophenyl ketone (Compound 128)

General procedure: 4

Starting compound I: compound 127 (0.07 g, 0.20 mmol)

Reaction time: 2 h

Purification: Chromatography using EtOAc

Product: Compound 128

¹³C NMR (CD₃OD): δ 181.3, 153.4, 136.6, 135.0, 128.7, 128.2, 126.1, 125.2, 124.0, 120.7, 116.3, 113.3, 36.6

### Example 29

3,5-dimethyl-4-isoxazolyl 4-(2-tolylamino)-2-chlorophenyl ketone (Compound 129)

General procedure: 1

Starting compound II: 4-bromo-3,5-dimethylisoxazol (0.35 g, 2.0 mmol)

Starting compound VIII: compound 6 (0.38 g, 2.2 mmol) in THF (5 mL)

Solvent: THF (3 mL)

Base: i-PrMgCl (2.2 mmol)

Metallation temperature: 0° C.

Metallation time: 1.5 h

Purification: Chromatography using CH₂Cl₂-EtOAc 20:1 followed by RP-HPLC

Product: Compound 129, 13 mg crystals (2%), the silyl group was lost upon work-up

¹³C NMR (CDCl₃): δ 188.0, 173.4, 159.6, 148.9, 138.0, 133.5, 132.4, 131.5, 131.4, 128.9, 127.2, 125.5, 123.8, 117.6, 115.5, 112.8, 17.9, 13.0, 11.4

### Example 30

4-(2-tolylamino)-2-chlorophenyl 4,5-dimethyl-2-thiazolyl ketone (Compound 130)

General procedure: 1

Starting compound II: 4,5-dimethylthiazol (0.21 mL, 2.0 mmol)

Starting compound VIII: compound 6 (0.38 g, 2.2 mmol) in THF (6 mL)

Solvent: THF (4 mL)

Base: n-BuLi (2.2 mmol)

Metallation temperature: −78° C.

Metallation time: 10 min

Purification: Chromatography using PE-EtOAc 2:1

Product: Compound 130, 0.64 g crystals (89%)

¹³C NMR (CDCl₃): δ 183.6, 162.9, 151.5, 149.1, 138.1, 135.8, 135.6, 134.3, 132.4, 131.3, 127.1, 125.4, 125.3, 123.9, 116.2, 111.7, 17.9, 15.0, 12.1

### Example 31

4-(2-tolylamino)-2-chlorophenyl 5-methyl-2-thienyl ketone (Compound 131)

General procedure: 1

Starting compound II: 2-methylthiophene (0.19 mL, 2.0 mmol)

Starting compound VIII: compound 6 (0.75 g, 2.0 mmol) in THF (5 mL)

Solvent: THF (4 mL)

Base: n-BuLi (2.2 mmol)

Metallation temperature: −78° C.

Metallation time: 10 min

Purification: Chromatography using PE-EtOAc 3:1

Product: Compound 131, 0.07 g crystals (10%), the silyl group was lost upon work-up

¹³C NMR (CDCl₃): δ 186.1, 150.7, 147.7, 142.1, 138.4, 135.7, 133.2, 131.6, 131.1, 131.0, 128.3, 126.9, 126.6, 124.7, 122.9, 115.8, 112.2, 17.7, 16.0

### Example 32

4-(2-benzonitrileamino)-2-chlorophenyl 3-chloro-2-thienyl ketone (Compound 132)

General procedure: 2

Starting compound VI: 2-fluorobenzonitrile (0.22 mL, 2.2 mmol)

Starting compound V: compound 2 (0.54 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 3:1

Product: Compound 132, 0.29 oil (39%)

¹³C NMR (CDCl₃): δ 185.6, 144.6, 144.1, 136.3, 134.1, 133.5, 133.3, 132.7, 132.5, 130.8, 130.7, 130.6, 121.8, 119.6, 117.1, 117.0, 116.9, 101.6

### Example 33

4-(2-trifluoromethylphenylamino)-2-chlorophenyl 3-chloro-2-thienyl ketone (Compound 133)

General procedure: 3

Starting compound VI: 2-bromotrifluoromethylbenzene (0.22 mL, 1.6 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 24 h

Reaction temperature: 100° C.

Purification: Chromatography using PE-EtOAc 4:1

Product: compound 133, 0.56 g oil (89%).

¹³C NMR (CDCl₃): δ 185.6, 146.2, 139.2, 136.4, 133.5, 133.0, 132.2, 131.2, 130.8, 130.5, 130.3, 127.2, 123.0, 121.8, 121.1, 120.9, 117.9, 115.1

### Example 34

4-(4-bromo-2-tolylamino)-2-chlorophenyl 3-chloro-2-thienyl ketone (Compound 134)

General procedure: 3

Starting compound VI: 5-bromo-2-iodotoluene (0.49 mg, 1.7 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 9 h

Reaction temperature: 100° C.

Purification: Chromatography using PE-EtOAc 6:1

Product: compound 134, 0.25 g oil (37%).

¹³C NMR (CDCl₃): δ 185.6, 148.0, 143.4, 137.6, 134.1, 133.8, 131.8, 131.6, 130.3, 130.1, 130.0, 129.0, 128.4, 124.6, 117.7, 115.9, 113.0, 17.8

### Example 35

4-(4-bromo-2-cyanophenylamino)-2-chlorophenyl 3-chloro-2-thienyl ketone (Compound 135)

General procedure: 2

Starting compound VI: 5-bromo-2-fluorobenzonitrile (0.42 mg, 2.1 mmol)

Starting compound V: compound 2 (0.54 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 3:1

Product: Compound 135, 0.19 g crystals (21%)

¹³C NMR (DMSO-d₆): δ 184.7, 146.0, 143.7, 137.3, 135.9, 135.4, 134.7, 131.5, 130.8, 130.6, 129.9, 129.3, 122.7, 117.3, 115.9, 115.1, 113.7, 105.5

### Example 36

4-(4-isoquinolylamino)-2-chlorophenyl 3-chloro-2-thienyl ketone (Compound 136)

General procedure: 3

Starting compound VI: 4-bromoisoquinoline (0.31 mg, 1.6 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 24 h

Reaction temperature: 100° C.

Purification: recrystallisation (EtOAc)

Product: compound 136, 0.28 g crystals (47%).

¹³C NMR (CDCl₃+4 dr. CD₃OD): δ 186.1, 149.3, 149.2, 137.2, 136.5, 133.8, 132.2, 132.2, 131.6, 131.0, 130.5, 130.3, 129.4, 128.9, 128.3, 128.1, 128.0, 122.0, 116.2, 113.1

### Example 37

4-(4-bromo-2,3,5,6-tetramethylphenylamino)-2-chlorophenyl 3-chloro-2-thienyl ketone (Compound 137)

General procedure: 3

Starting compound VI: dibromodurene (0.48 mg, 1.7 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 3 days

Reaction temperature: 100° C.

Purification: Chromatography using PE-EtOAc 5:1 followed by recrystallisation (EtOAc)

Product: compound 137, 0.01 g crystals (1.5%).

¹H NMR (CDCl₃+6 dr. CD₃OD): δ 7.58 (1H,d), 7.29 (1H,d), 7.00 (1H,d), 6.44 (1H,Br), 6.38 (1H,d,Br), 2.46 (6H,s), 2.20 (6H,s)

### Example 38

4-(4-bromo-2-chlorophenylamino)-2-chlorophenyl 3-chloro-2-thienyl ketone (Compound 138)

General procedure: 3

Starting compound VI: 4-bromo-2-chloroiodobenzene (0.52 mg, 1.7 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 10 h

Reaction temperature: 100° C.

Purification: Chromatography using PE-EtOAc 10:1

Product: compound 138, 0.16 g crystals (23%).

¹³C NMR (CDCl₃): δ 185.6, 145.0, 137.1, 133.5, 132.6, 132.4, 131.4, 131.1, 130.7, 130.6, 130.5, 130.4, 124.7, 119.6, 118.4, 115.6, 114.1

### Example 39

4-(4-bromo-2-nitrophenylamino)-2-chlorophenyl 3-chloro-2-thienyl ketone (Compound 139)

General procedure: 2

Starting compound VI: 1-bromo-4-fluoro-3-nitrobenzene (0.19 mL, 2.1 mmol)

Starting compound V: compound 2 (0.54 g9 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 5:1

Product: Compound 139, 0.25 g crystals (17%)

¹H NMR (CDCl₃): δ 9.39 (1H,s), 8.37 (1H,d), 7.67 (1H,d), 7.57 (1H,dd), 7.46 (1H,dd), 7.34 (1H,d), 7.30 (1H,d), 7.23 (1H,dd), 7.05 (1H,d)

### Example 40

4-(2-amino-4-bromophenylamino)-2-chlorophenyl 3-chloro-2-thienyl ketone (Compound 139)

General procedure: 4

Starting compound I: compound 139 (0.94 g, 2.0 mmol)

Reaction time: 1 h

Purification: Chromatography using EtOAc-PE 1:1

Product: Compound 140, 0.62 g oil (70%).

¹H NMR (CDCl₃): δ 7.58 (d,1H), 7.32 (d,1H), 7.00 (m,3H), 6.88 (dd,1H), 6.68 (d,1H), 6.60 (dd,1H), 5.45 (s,Br,1H), 3.87 (s,Br,2H)

### Example 41

4-phenylamino-2-chlorophenyl 3-chloro-2-thienyl ketone (Compound 141)

General procedure: 3

Starting compound VI: bromobenzene (0.27 mg, 1.7 mmol)

Starting compound V: compound 2 (0.41 g, 1.5 mmol)

Solvent: 1,4-dioxane (4 mL)

Base: Cs₂CO₃ (0.68 g, 2.1 mmol)

Reaction time: 16 h

Reaction temperature: 100° C.

Purification: Chromatography using PE-EtOAc 10:1

Product: compound 141, oil

¹H NMR (CDCl₃): δ 7.58 (1H,d), 7.4-7.3 (3H,m), 7.18 (2H,m), 7.10 (1H,m), 7.03 (1H,d), 7.00 (1H,d), 6.89 (1H,dd), 6.00 (1H,s)

### Example 42

4-(4-bromo-2-trifluoromethylphenylamino)-2-chlorophenyl 3-chloro-2-thienyl ketone (Compound 142)

General procedure: 2

Starting compound VI: 5-bromo-2-fluorobenzotrifluoride (0.50 g, 2.1 mmol)

Starting compound V: compound 2 (0.54 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 3:1

Product: Compound 142, 0.07 oil (7%)

¹H NMR (CDCl₃): δ 7.76 (1H,d), 7.62 (1H,d), 7.60 (1H,m), 7.38 (1H,d), 7.35 (1H,d), 7.06 (1H,d), 7.03 (1H,d), 6.94 (1H,dd), 6.11 (1H,s)

### Example 43

4-(3-pyridylamino)-2-chlorophenyl 3-chloro-2-thienyl ketone (Compound 143)

General procedure: 3

Starting compound VI: 3-bromopyridine (0.22 mL, 1.1 mmol)

Starting compound V: compound 2 (0.27 g, 1.0 mmol)

Solvent: 1,4-dioxane (4 mL)

Base: t-BuOK (0.16 g, 1.4 mmol)

Reaction time: 16 h

Reaction temperature: 100° C.

Purification: Chromatography using EtOAc

Product: compound 143, 0.03 g oil (7%)

¹³C NMR (CDCl₃): δ 185.7, 146.5, 144.3, 142.5, 137.5, 136.4, 133.7, 132.2, 131.4, 130.5, 130.3, 130.2, 126.9, 124.0, 116.7, 113.8

### Example 44

2-chloro-4-nitrobenzoylpyrrole Compound 144

General procedure: 1

Starting compound II: pyrrole (0.14 mL, 2.0 mmol)

Starting compound III: 2-chloro-4-nitrobenzoyl chloride (0.48 g, 2.2 mmol) in THF (1 mL)

Solvent: THF (2 mL)

Base: NaH (3.0 mmol)

Metallation temperature: 0° C.

Metallation time: 30 min

Purification: Chromatography using CH₂Cl₂-EtOAc 3:1

Product: Compound 144, 0.26 g crystals (53%)

¹³C NMR (CDCl₃): δ 163.3, 149.3, 139.4, 133.1, 129.8, 125.4, 122.0, 120.1, 114.8

### Example 45

4-amino-2-chlorobenzoylpyrrole Compound 145

General procedure: 4

Starting compound IV: compound 144 (0.50 g, 2.0 mmol)

Reaction time: 1 h

Purification: Chromatography using EtOAc-PE 1:1

Product: Compound 145, 0.20 g crystals (46%).

¹H NMR (CDCl₃): δ 7.25 (d,1H), 7.16 (t,2H), 6.73 (d,1H), 6.58 (dd,1H), 6.30 (t,2H), 4.06 (s,Br,2H)

### Example 46

1-[4-(2-tolylamino)-2-chlorobenzoyl]pyrrole (Compound 146)

General procedure: 3

Starting compound VI: bromotoluene (0.29 mg, 1.7 mmol)

Starting compound V: compound 145 (0.33 g, 1.5 mmol)

Solvent: 1,4-dioxane (4 mL)

Base: Cs₂CO₃ (0.68 g, 2.1 mmol)

Reaction time: 18 h

Reaction temperature: 100° C.

Purification: Chromatography using PE-EtOAc 10:1

Product: compound 146, 0.13 g oil (29%)

¹H NMR (CDCl₃): δ 7.25 (m,4H), 7.18 (t,2H), 7.11 (m,1H), 6.82 (d,1H), 6.69 (dd,1H), 6.29 (t,2H), 5.80 (s,Br,1H), 2.24 (s,3H)

### Example 47

2-chloro-4-nitrophenyl 3,5-dimethyl-2-pyrrolyl ketone Compound 147

General procedure: 1

Starting compound II: 2,4-dimethylpyrrole (0.21 mL, 2.0 mmol)

Starting compound III: 2-chloro-4-nitrobenzoyl chloride (0.48 g, 2.2 mmol) in THF (1 mL)

Solvent: THF (2 mL)

Base: NaH (3.0 mmol)

Metallation temperature: 0° C.

Metallation time: 30 min

Purification: recrystallisation (EtOAc)

Product: Compound 147, 0.18 g crystals (32%)

¹³C NMR (CDCl₃): δ 179.7, 148.5, 145.9, 138.7, 132.9, 132.2, 129.0, 127.3, 125.2, 122.2, 113.9, 13.3, 12.9

### Example 48

4-amino-2-chlorophenyl 3,5-dimethyl-2-pyrrolyl ketone (Compound 148)

General procedure: 4

Starting compound IV: compound 147 (0.53 g, 2.0 mmol)

Reaction time: 1.5 h

Purification: Chromatography using EtOAc-PE 1:1

Product: Compound 148, 0.07 g oil (14%).

¹H NMR (CDCl₃): δ 9.61 (s,Br,1H), 7.12 (d,1H), 6.70 (d,1H), 6.57 (dd,1H), 5.81 (d,1H), 3.99 (s,Br,2H)

### Example 49

4-(2-tolylamino)-2-chlorophenyl 3,5-dimethyl-2-pyrrolyl ketone (Compound 149)

General procedure: 3

Starting compound VI: bromotoluene (0.29 mg, 1.7 mmol)

Starting compound V: compound 148 (0.37 g, 1.5 mmol)

Solvent: 1,4-dioxane (4 mL)

Base: Cs₂CO₃ (0.68 g, 2.1 mmol)

Reaction time: 24 h

Reaction temperature: 100° C.

Purification: Chromatography using PE-EtOAc 3:1

Product: compound 149, 49 mg oil (10%)

¹H NMR (CDCl₃): δ 9.07 (s,Br,1H), 7.25 (m,3H), 7.19 (d,1H), 7.07 (m,1H), 6.86 (d,1H), 6.75 (dd,1H), 5,84 (d,1H), 5.57 (s,Br,1H), 2.28 (s,3H), 2.25 (s,3H), 1.84 (s,3H)

### Example 50

4-(2-amino-4-trifluoromethylphenylamino)-2-chlorophenyl 3-chloro-2-thienyl ketone (Compound 150)

General procedure: 2

Starting compound VI: 4-fluoro-3-nitrobenzotrifluoride (0.29 mL, 2.1 mmol)

Starting compound V: compound 2 (0.54 g, 2.0 mmol)

Solvent: DMSO (10 mL)

Base: t-BuOK (0.45 g, 2 mmol)

Reaction time: 24 h

Reaction temperature: rt

Purification: Chromatography using EtOAc-PE 10:1 (0.15 g oil)

Followed by

General procedure: 4

Starting compound IV: above compound (0.15 g, 0.3 mmol)

Reaction time: 1.5 h

Purification: Chromatography using EtOAc-PE 1:6

Product: Compound 150, 25 mg oil (3%).

¹³C NMR (CDCl₃): δ 185.7, 147.6, 141.8, 136.5, 133.8, 132.0, 131.5, 130.4, 130.2, 129.3, 128.6, 124.9, 124.1, 116.0, 113.1

### Example 51

4-(2-tolylamino)-2-chlorophenyl 3-chloro-4-pyridyl ketone (Compound 151)

General procedure: 1

Starting compound II: 3-chloropyridine (0.18 mL, 2.0 mmol)

Starting compound VIII: compound 7 (0.46 g, 1.0 mmol) in THF (2 mL)

Solvent: THF (6 mL)

Base: LDA (2.2 mmol)

Metallation temperature: −78° C.

Metallation time: 3.5 h

Purification: Chromatography using PE-EtOAc 3:1 followed by RP-HPLC

Product: Compound 151, 16 mg crystals (2%). The silyl group was lost upon work-up.

¹³C NMR (CDCl₃): δ 189.1, 151.2, 149.2, 148.4, 146.2, 137.1, 136.8, 135.0, 133.3, 131.5, 127.3, 126.5, 125.0, 124.1, 123.3, 115.5, 112.3, 17.9

### Example 52

5-chloro-3-methyl-2-benzothienyl 4-(2-tolylamino)-2-chlorophenyl ketone (Compound 152)

General procedure: 1

Starting compound II: 5-chloro-3-methylbenzothiophene (0.37 mg, 2.0 mmol)

Starting compound VIII: compound 7 (0.46 g, 1.0 mmol) in THF (2 mL)

Solvent: THF (4 mL)

Base: n-BuLi (2.2 mmol)

Metallation temperature: −78° C.

Metallation time: 10 min

Purification: Chromatography using PE-EtOAc 8:1 affords 98 mg (8%) of the protected derivative.

¹³C NMR (CDCl₃): δ 188.8, 154.2, 142.5, 141.6, 139.1, 138.9, 137.6, 137.4, 132.9, 131.8, 131.2, 130.7, 130.6, 128.6, 127.4, 127.0, 126.9, 123.6, 123.2, 118.4, 115.0, 18.9, 18.4, 14.2, 13.1

Deprotection following general procedure 5

Purification: Chromatography using PE-EtOAc 3:1

Product: Compound 152, 20 mg oil (33%).

¹H NMR (CDCl₃): δ 7.80 (d,1H), 7.73 (d,1H), 7.41 (dd,1H), 7.38 (d,1H), 7.26 (m,3H), 7.12 (m,1H), 6.83 (d,1H), 6.71 (dd,1H), 5.73 (s,Br,1H), 2.50 (s,3H), 2.27 (s,3H)

### Example 53

2-benzofuranyl 4-(2-tolylamino)-2-chlorophenyl ketone (Compound 153)

General procedure: 1

Starting compound II: benzofuran (0.22 mL, 2.0 mmol)

Starting compound VIII: compound 7 (0.46 g, 1.0 mmol) in THF (2 mL)

Solvent: THF (4 mL)

Base: n-BuLi (2.2 mmol)

Metallation temperature: −78° C.

Metallation time: 10 min

Purification: Chromatography using PE-EtOAc 20:1 affords 0.34 g (33%) of the protected derivative.

¹H NMR (CDCl₃): δ 7.66 (1H, d), 7.60 (1H, d), 7.46 (1H, t), 7.39 (1H, d), 7.35-7.14 (6H,m), 6.68 (1H, d), 6.47 (1H, dd), 2.20 (3H, s), 1.45 (3H, m), 1.09 (18H, d)

Deprotection following general procedure 5

Purification: Chromatography using PE-EtOAc 3:1

Product: Compound 153, 136 mg (57%).

¹H NMR (DMSO): δ 8.40 (1H, s), 7.83 (1H, d), 7.73 (1H, d), 7.61 (1H, s), 7.58 (1H, d), 7.56 (1H, dt), 7.38 (1H, t), 7.35-7.20 (3H, m), 7.13 (1H, dt), 6.84 (1H, d), 6.76 (1H, dd), 2.34 (3H, s)

### Example 54

3-benzothienyl 4-(2-tolylamino)-2-chlorophenyl ketone (Compound 154)

General procedure: 1

Starting compound II: 3-bromobenzothiophene (0.28 mL, 2.0 mmol)

Starting compound VIII: compound 7 (0.46 g, 1.0 mmol) in THF (2 mL)

Solvent: THF (4 mL)

Base: n-BuLi (2.2 mmol)

Metallation temperature: −78° C.

Metallation time: 10 min

Purification: Chromatography using PE-EtOAc 20:1 affords 0.41 mg (34%) of the protected derivative.

¹H NMR (CDCl₃): δ 7.87 (d,1H), 7.84 (d,1H), 7.66 (d,1H), 7.44 (t,1H), 7.37 (d,1H), 7.35 (d,1H), 7.28 (m,4H), 6.68 (d,1H), 6.48 (dd,1H), 2.20 (s,3H), 1.45 (m,3H), 1.09 (d,18H)

Deprotection following general procedure 5

Purification: recrystallisation EtOAc

Product: Compound 154, 0.14 g yellow crystals (55%).

¹H NMR (DMSO): δ 8.34 (1H, s), 8.08 (1H, d), 8.03 (1H, d), 7.92 (1H, s), 7.60-7.50 (2H, m), 7.46 (1H, t), 7.35-7.20 (3H, m), 7.12 (1H, dt), 6.86 (1H, d), 6.78 (1H, dd), 2.24 (3H, s)

### Example 55

3,5-dimethyl-2-benzothienyl 4-(2-tolylamino)-2-chlorophenyl ketone (Compound 155)

General procedure: 1

Starting compound II: 3,5-dimethylbenzothiophene (0.23 mL, 2.0 mmol)

Starting compound VIII: compound 7 (0.46 g, 1.0 mmol) in THF (2 mL)

Solvent: THF (4 mL)

Base: n-BuLi (2.2 mmol)

Metallation temperature: −78° C.

Metallation time: 10 min

Purification: Chromatography using PE-CH₂Cl₂ 20:1 affords 0.20 g (18%) of the protected derivative.

¹H NMR (CDCl₃): δ 7.68 (d,1H), 7.60 (s,1H), 7.24 (m,6H), 6.63 (d,1H), 6.51 (dd,1H), 2.48 (s,3H), 2.45 (s,3H), 2.18 (s,3H), 1.45 (m,3H), 1.08 (d,18H)

Deprotection following general procedure 5

Purification: Chromatography using PE-EtOAc 7:1

Product: Compound 155, 0.10 g (71%).

¹H NMR (CDCl₃): δ 7.70 (1H, d), 7.63 (1H, s), 7.38 (1H, d), 7.35-7.18 (4H, m), 7.11 (1H, dt), 6.84 (1H, d), 6.72 (1H, dd), 2.54 (3H, s), 2.50 (3H, s), 2.27 (3H, s)

### Example 56

4-(2-tolylamino)-2-chlorophenyl 3-chloro-2-pyrazinyl ketone (Compound 156)

General procedure: 1

Starting compound II: 2-chloropyrazine (0.18 mL, 2.0 mmol)

Starting compound VIII: compound 7 (0.46 g, 1.0 mmol) in THF (2 mL)

Solvent: THF (10 mL)

Base: LiTMP (2.6 mmol)

Metallation temperature: −78° C.

Metallation time: 45 min

Purification: Chromatography using PE-EtOAc 8:1 affords 15 mg (1.5%) of the protected derivative as a yellow oil.

¹³C NMR (CDCl₃): δ 188.6, 156.4, 152.5, 143.8, 141.9, 141.3, 137.2, 135.9, 133.6, 131.8, 130.8, 127.3, 127.1, 123.5, 117.9, 115.0, 22.4, 18.8, 14.0

Deprotection following general procedure 5

Purification: Chromatography using PE-EtOAc 3:1

Product: Compound 156, 2 mg (40%).

¹H NMR (CDCl₃): δ 8.51 (1H, d), 8.45 (1H, d), 7.80 (1H, d), 7.35-7.13 (4H, m), 6.72 (1H, dd), 6.70 (1H, d), 5.88 (1H, bs), 2.25 (3H, s)

### Example 57

2-chloro-4-nitrobenzoylindole (Compound 157)

General procedure: 1

Starting compound II: indole (0.23 mg, 2.0 mmol)

Starting compound III: 2-chloro-4-nitrobenzoyl chloride (0.48 g, 2.2 mmol) in THF (1 mL)

Solvent: THF (2 mL)

Base: NaH (3.0 mmol)

Metallation temperature: 0° C.

Metallation time: 30 min

Purification: Chromatography using PE-EtOAc 10:1

Product: Compound 157, 0.20 g (33%)

¹³C NMR (CDCl₃): δ 163.8, 149.2, 140.4, 135.4, 132.8, 131.0, 129.7, 125.8, 125.6, 125.4, 125.0, 122.3, 121.3, 116.5, 111.0

### Example 58

4-amino-2-chlorobenzoylindole (Compound 158)

General procedure: 4

Starting compound IV: compound 157 (0.30 g, 1.0 mmol)

Reaction time: 1 h

Purification: Chromatography using EtOAc-PE 1:2

Product: Compound 158, 0.19 g (74%).

¹H NMR (CDCl₃): δ 8.34 (1H, d), 7.57 (1H, d), 7.36 (1H, dt), 7.29 (1H, dt), 7.29 (1H, d), 7.13 (1H, d), 6.75 (1H, d), 6.62 (1H, dd), 6.58 (1H, d), 4.0 (2H, bs)

### Example 59

1-[4-(2-tolylamino)-2-chlorobenzoyl]indole (Compound 159)

General procedure: 3

Starting compound VI: bromotoluene (0.13 mg, 0.77 mmol)

Starting compound V: compound 158 (0.19 g, 0.70 mmol)

Solvent: 1,4-dioxane (4 mL)

Base: Cs₂CO₃ (0.68 g, 2.1 mmol)

Reaction time: 18 h

Reaction temperature: 100° C.

Purification: Chromatography using PE-EtOAc 10:1 followed by RP-HPLC

Product: compound 146, 7 mg (3%)

¹H NMR (CDCl₃): δ 8.38 (1H, d), 7.58 (1H, d), 7.44-7.20 (6H, m), 7.16 (1H, d), 7.13 (1H, dt), 6.86 (1H, d), 6.76 (1H, dd), 6.59 (1H, d), 5.6 (1H, bs), 2.28 (3H, s)

### Example 60

4-(2-tolylamino)-2-chlorophenyl 2,5-dichloro-3-thienyl ketone (Compound 160)

General procedure: 1

Starting compound II: 3-bromo-2,5-dichlorothiophene (0.18 mL, 2.0 mmol)

Starting compound VIII: compound 7 (0.46 g, 1.0 mmol) in THF (2 mL)

Solvent: THF (4 mL)

Base: n-BuLi (2.2 mmol)

Metallation temperature: −78° C.

Metallation time: 10 min

Purification: Chromatography using PE-CH₂Cl₂ 20:1 affords 0.24 g (22%) of the protected derivative as a yellow oil.

¹³C NMR (CDCl₃): δ 186.2, 154.7, 142.4, 137.5, 137.4, 133.7, 131.8, 131.6, 131.3, 131.1, 127.2, 127.0, 126.9, 126.7, 126.3, 118.4, 115.0, 18.8, 18.4, 14.2

Deprotection following general procedure 5

Purification: Chromatography using PE-EtOAc 10:1

Product: Compound 160, 104 mg (70%).

¹H NMR (CDCl₃): δ 7.38 (1H, d), 7.32-7.19 (3H, m), 7.14 (1H, dt), 7.00 (1H, s), 6.78 (1H, d), 6.69 (1H, dd), 5.75 (1H, bs), 2.25 (3H, s)

### Example 61

5-indolyl 4-(2-tolylamino)-2-chlorophenyl ketone (Compound 161)

5-Bromo-1-triisopropylsilylindole: NaH (6 mmol) is added to a solution of 5-bromoindole (4 mmol) in dry DMF (4 mL) at 0° C. Stirring at 0° C. for 30 min before addition of triisopropylsilylchloride. Stirring overnight a rt. Addition of water, extraction with Et₂O (3×). The combined organic layers are washed with brine, dried over MgSO₄, filtered and concentrated in vacuo. Chromatography using PE-EtOAc 20:1 affords 5-bromo-1-triisopropylsilylindole (0.36 g, 25%). ¹H NMR (CDCl₃): δ 7.73 (1H, d), 7.36 (1H, d), 7.23 (1H, d), 7.21 (1H, dd), 6.54 (1H, dd), 1.67 (3H, m), 1.13 (18H, d)

General procedure: 1

Starting compound II: 5-bromo-1-triisopropylsilylindole (0.36 mg, 1.0 mmol)

Starting compound VIII: compound 7 (0.23 g, 0.5 mmol) in THF (1 mL)

Solvent: THF (2 mL)

Base: n-BuLi (1.1 mmol)

Metallation temperature: −78° C.

Metallation time: 15 min

Purification: Chromatography using PE-EtOAc 20:1 affords 90 mg (14%) of the protected derivative.

¹H NMR (CDCl₃): δ 8.05 (1H, d), 7.70 (1H, dd), 7.50 (1H, d), 7.32-7.16 (6H, m), 6.70 (1H, d), 6.65 (1H, d), 6.50 (1H, dd), 2.21 (3H, s), 1.69 (3H, m), 1.45 (3H, m), 1.13 (18H, d), 1.09 (18H, d)

Deprotection following general procedure 5

Purification: Chromatography using PE-EtOAc 3:1

Product: Compound 161, 40 mg (82%).

¹H NMR (CDCl₃): δ 8.65 (1H, bs), 8.10 (1H, d), 7.81 (1H, dd), 7.42 (1H, d), 7.34-7.16 (5H, m), 7.08 (1H, dt), 6.88 (1H, d), 6.74 (1H, dd), 6.62 (1H, m), 5.66 (1H, bs), 2.28 (3H, s)

### Example 62

4-(2-tolylamino)-2-chlorophenyl 3-chloro-1-oxidopyridin-2-yl ketone (Compound 162)

Compound 123 (16 mg) and m-chloroperbenzoic acid (1,8 equiv) are stirred in CH₂Cl₂ (3 mL) at rt overnight. Addition of Na₂S₂O₃, stirring for 15 min, filtration. Addition of K₂CO₃, stirring for 1 h, drying over MgSO₄, filtration, concentration in vacuo.

Purification: Chromatography using EtOAc-CH₃OH 15:1

Product: Compound 162, 3 mg (17%).

¹H NMR (CDCl₃): δ 8.12 (1H, d), 7.93 (1H, d), 7.35 (1H, d), 7.32-7.12 (5H, m), 6.73-6.64 (2H, m), 6.02 (1H, bs), 2.23 (3H, s)

### Example 63

4-[methyl(2-tolyl)amino]-2-chlorophenyl 3-chloro-2-thienyl ketone (Compound 163)

Compound 101 (400 mg, 1.1 mmol) was dissolved in dry THF under argon and cooled to 0° C. Lithium bis (trimethylsilyl)amide (1M in hexane, 1.5 eq.) was added dropwise. Stirring for 20 min before addition of methyliodide (1.5 eq.). Stirring for 30 min. Addition of sat. aq. NH₄Cl, the aqueous phase was extracted with EtOAc (3 times). The combined extracts were dried (MgSO₄), filtered and evaporated to afford the crude product.

Purification: Chromatography using EtOAc-PE 1:10

Product: Compound 163, 230 mg (56%).

¹³C NMR (CDCl₃): δ 185.7, 151.9, 144.7, 137.0, 136.4, 134.2, 131.9, 131.8, 131.1, 130.0, 129.3, 128.3, 127.9, 127.7, 125.8, 113.2, 110.1, 39.1, 17.7

### Example 64

Tablet containing compound 101

| | |
|---|---|
| Compound 101 (active substance) | 50 mg |
| Lactose | 125 mg |
| Starch | 12 mg |
| Methyl cellulose | 2 mg |
| Sodium carboxymethyl cellulose | 10 mg |
| Magnesium stearate | 1 mg |

The active substance, lactose and starch are mixed to a homogeneous state in a suitable mixer and moistened with a 5 per cent aqueous solution of methyl cellulose 15 cps. The mixing is continued until granules are formed. If necessary, the wet granulation is passed through a suitable screen and dried to a water content of less than 1% in a suitable drier, e.g. fluid bed or drying oven. The dried granules are passed through a 1 mm screen and mixed to a homogeneous state with sodium carboxymethyl cellulose. Magnesium stearate is added, and the mixing is continued for a short period of time. Tablets with a weight of 200 mg are produced from the granulation by means of a suitable tabletting machine.

### Example 65

Formulation for injection containing compound 101

| | |
|---|---|
| Compound 101 (active substance) | 1% |
| Sodium chloride | q.s. |
| Ethanol | 10% |
| Water for injection to make | 100% |

The active substance is dissolved in ethanol (10%) then water for injection made isotonic with sodium chloride is added to make 100%. The mixture is filled into ampoules and sterilized.

### Example 66

Cream formulation containing compound 101

Compound 101 (10 g) was dissolved in Octyldodecyl myristate (250 g) to form Part A. Methylparaben (1 g) and propylparaben (0.2 g) were dissolved in phenoxyethanol (6 g) and mixed with a 0.025 M Phosphate buffer pH=7.5 (632,8 g) to form Part B. Cetostearyl alcohol (50 g) and ARLACEL 165® (50 g) was melted in a vessel at 700 to 80° C. Part A was added and heated to 60-70° C. The aqueous phase was likewise heated to 60-70° C. and slowly added to the melted oil phase under high speed stirring. The homogenized components were cooled to room temperature.

1. A compound of general formula I 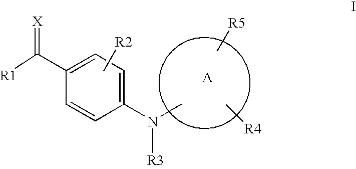
wherein
R₁ is a heteroaromatic ring system comprising 1-4 heteroatoms, optionally substituted by one or more, same or different substituents selected from the group consisting of hydrogen, halogen, haloalkyl, hydroxy, hydroxyalkyl, hydroxyalkyloxy, mercapto, cyano, carboxy, nitro, alkyl, alkenyl, alkynyl, cycloalkyl, cycloalkenyl, heterocycloalkyl, aryl, heteroaryl, aralkyl, alkylaryl, alkoxy, aralkoxy, alkylthio, alkoxycarbonyl, alkylcarbonyloxy, alkoxycarbonyloxy, alkylsulfonyloxy, alkyloxysulfonyl, alkylcarbonylamino, aminocarboaminoalkyl, aminosulfonyl, alkylsulfonylamino, alkanoyl, alkylcarbonyl, —NR₆R₇ or —CONR₆R₇, wherein R₆ and R₇ are the same or different and individually represent hydrogen, alkyl, aryl or —(Z—O)ₙ—Z, wherein Z is alkyl and n is an integer from 1 to 7;;
X is O, S, N—OH or NR₈, wherein R₈ is hydrogen or alkyl;
R₂ is hydrogen, halogen, haloalkyl, hydroxy, hydroxyalkyl, mercapto, cyano, nitro, alkyl, alkenyl, alkynyl, cycloalkyl, aryl, aralkyl, alkylaryl, alkoxy, aralkoxy, alkylthio, alkoxycarbonyl, alkylcarbonylamino, alkylcarbonyloxy, alkoxycarbonyloxy, alkylcarbonyl —NR₆R₇ or —CONR₆R₇;
R₃ is hydrogen, alkyl, alkenyl, alkynyl, cycloalkyl, cycloalkenyl, carboxy or aryl;
A is aryl or a heteroaromatic ring system comprising 1-4 heteroatoms;
R₄ is hydrogen, halogen, haloalkyl, hydroxy, hydroxyalkyl, mercapto, cyano, carboxy, nitro, alkyl, alkenyl, alkynyl, cycloalkyl, heterocycloalkyl, cycloalkenyl, aryl, heteroaryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkyloxysulfonyl, alkylcarbonyl, —NR₆R₇ or —CONR₆R₇, wherein R₆ and R₇ are the same or different and individually represent hydrogen, alkyl, aryl or —(Z—O)ₙ—Z; —N—(C═O)—CF₃, —N—Q—Y, —N—(COO)—Q—Y or —N—(C═O)—N—Q—Y, wherein Q is a bond, —CO—, —CS—, —SO₂— or —CR₉R₁₀—(O—C═O)—, wherein R₉ and R₁₀ are the same or different and individually represent hydrogen, alkyl or aryl, and Y is alkyl, alkenyl, alkynyl, cycloalkyl, cycloalkenyl or aryl, optionally substituted by hydrogen, halogen, haloalkyl, hydroxy, hydroxyalkyl, mercapto, cyano, carboxy, nitro, alkyl, alkenyl, alkynyl, cycloalkyl, cycloalkenyl, aryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkoxysulfonyl, alkylcarbonyl, —NR₆R₇ or —CONR₆R₇, wherein R₆ and R₇ are the same or different and individually represent hydrogen, alkyl or aryl, or —(Z—O)ₙ—Z;
R₅ is hydrogen, halogen, haloalkyl, hydroxy, hydroxyalkyl, mercapto, cyano, carboxy, carbamoyl, amino, nitro, alkyl, alkenyl, alkynyl, cycloalkyl, heterocycloalkyl, cycloalkenyl, aryl, heteroaryl, aralkyl, alkylaryl, alkoxy, aralkoxy, alkylthio, alkoxycarbonyl, alkylcarbonylamino, aminocarboaminoalkyl, aminosulfonyl, alkylsulfonylamino, alkylcarbonyloxy, alkoxycarbonyloxy, alkylsulfonyloxy, alkoxysulfonyl, alkylcarbonyl, —NR₆R₇ or —CONR₆R₇, wherein R₆ and R₇ are the same or different and individually represent hydrogen, alkyl or aryl;
and pharmaceutically acceptable salts, hydrates, solvates or esters thereof.

2. A compound according to claim 1, wherein R , is an optionally substituted, mono- or bicyclic heteroaromatic ring system comprising 1-4 heteroatoms, each ring comprising 5 or 6 ring atoms.

3. A compound according to claim 1 or 2, wherein the heteroaromatic ring system is selected from the group consisting of thienyl, furyl, benzofuranyl, isobenzofuranyl, pyrrolyl, imidazolyl, benzimidazolyl, pyrazolyl, thiazolyl, isothiazolyl, isoxazolyl, pyridyl, pyrazinyl, pyrimidinyl, pyridazinyl, indolizinyl, isoindolyl, indolyl, indazolyl, purinyl, quinolyl, isoquinolyl, phthalazinyl, quinazolinyl, benzothienyl, isobenzothienyl, benzothiazolyl, benzoisothiazolyl, triazolyl, tetrazolyl, quinoxalyl, allopurinyl, benzotriazolyl or oxazolyl.

4. A compound according to claim 3, wherein the heteroaromatic ring system comprises 1 heteroatom.

5. A compound according to claim 4, wherein the heteroaromatic ring system is selected from the group consisting of thienyl, furyl, pyrrolyl, pyridyl, benzofuranyl, benzothienyl, indolyl, isoindolyl, quinolyl, isoquinolyl, isobenzothienyl or isobenzofuryl.

6. A compound according to claim 4, wherein the heteroatom is sulphur or nitrogen.

7. A compound according to claim 6, wherein the heteroaromatic ring system is selected from the group consisting of thienyl, benzothienyl,isobenzothienyl and pyridyl.

8. A compound according to any of claims 1-7, wherein the heteroaromatic ring system R₁ is substituted by one or more of the same or different substituents selected from the group consisting of halogen, hydroxy, hydroxyalkyl, trifluoromethyl, C₁₋₄ alkyl, C₂₋₄ alkenyl, C₂₋₄ alkynyl, C₁₋₄ alkoxycarbonyl, cyano, —NR₆R₇ or —CONR₆R₇, wherein R₆ and R₇ are as indicated in claim 1.

9. A compound according to any of claims 1-8, wherein R₂ is hydrogen, hydroxy, halogen, mercapto, trifluoromethyl, amino, C₁₋₆ alkyl, C₂₋₆ alkenyl, C₂₋₆ alkynyl, C₁₋₆ alkoxy, C₁₋₆ alkylthio, C₁₋₆ alkylamino, C₁₋₆ alkoxycarbonyl, cyano, —CONH₂, aryl or nitro.

10. A compound according to claim 9, wherein R₂ is hydrogen, halogen, hydroxy, trifluoromethyl, amino, C₁₋₄ alkyl, C₂₋₄ alkenyl or C₁₋₄ alkoxy.

11. A compound according to any of claims 1-10, wherein R₃ is hydrogen, C₁₋₆ alkyl, C₁₋₆ alkenyl or aryl.

12. A compound according to any of claims 1-11, wherein R₄ is halogen, CN, CF₃ C1₋₆ alkyl, —NH₂ or —N—(C═O)—CF₃.

13. A compound according to any of claims 1-11, wherein R₄ is —N—Q—Y, wherein Q is a bond —CO— or —CS—, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkyl or aryl.

14. A compound according to any of claims 1-11, wherein R₄ is —N—(COO)—Q—Y, wherein Q is a bond or —CR₉R₁₀—(O—C═O)—, wherein R₉ and R₁₀ are the same or different and individually represent hydrogen, trifluoromethyl or C₁₋₆ alkyl, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkenyl or aryl.

15. A compound according to any of claim 1-11, wherein R₄ is —N—(C═O)—N—Q—Y, wherein Q is a bond or —CR₉R₁₀—(O—C═O)—, wherein R₉ and R₁₀ are the same or different and individually represent hydrogen, trifluoromethyl or C₁₋₆ alkyl, and Y is optionally substituted C₁₋₁₅ alkyl, C₂₋₁₅ alkenyl, C₂₋₁₅ alkynyl, C₃₋₁₀ cycloalkyl, C₃₋₁₀ cycloalkenyl, aryl or —(Z—O)ₙ—Z, wherein Z and n are as indicated in claim 1.

16. A compound according to any of claims 1-15, wherein R₅ is hydrogen, halogen, hydroxy, mercapto, trifluoromethyl, amino, C₁₋₆ alkyl, C₂₋₆ alkenyl, C₂₋₆ alkynyl, C₁₋₆ alkoxy, C₁₋₆ alkylthio, C₁₋₆ alkylamino, C₁₋₆ alkoxycarbonyl, aryl, cyano, carboxy or carbamoyl.

17. A compound according to any of claims 1-16, wherein X is O.

18. A compound according to any of claims 1-17, wherein A is phenyl.

19. A compound according to claim 1 which has the general formula Ia 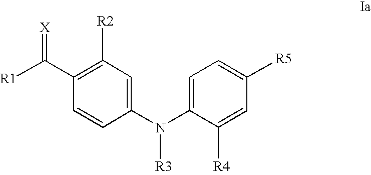
wherein R₁, R₂, R_{3,} R_{4,} R₅ and X are as indicated in claim 1.

20. A compound according to any of claims 1-19 selected from the group consisting of
4-(2-tolylamino)-2-chlorophenyl 3-chloro-2-thienyl ketone (compound 101)
4-(2-nitrophenylamino)-2-chlorophenyl 3-chloro-2-thienyl ketone (compound 102)
4-(2-aminophenylamino)-2-chlorophenyl 3-chloro-2-thienyl ketone (compound 103)
4-(2-tolylamino)-2-chlorophenyl 3-methyl-2-thienyl ketone (compound 106)
4-(2-nitrophenylamino)-2-chlorophenyl 3-methyl-2-thienyl ketone (compound 107)
4-(2-aminophenylamino)-2-chlorophenyl 3-methyl-2-thienyl ketone (compound 108)
4-(2-nitrophenylamino)-2-chlorophenyl 1-methyl-2-pyrrolyl ketone (compound 111)
4-(2-aminophenylamino)-2-chlorophenyl 1-methyl-2-pyrrolyl ketone (compound 112)
4-(2-tolylamino)-2-chlorophenyl 1-methyl-2-pyrrolyl ketone (compound 113)
4-(2-tolylamino)-2-chlorophenyl 1,3,5-trimethyl-4-pyrazolyl ketone (compound 114)
3-methyl-2-benzothienyl 4-(2-tolylamino)-2-chlorophenyl ketone (compound 115)
4-(2-tolylamino)-2-chlorophenyl 4-methyl-3-thienyl ketone (compound 116)
4-(2-tolylamino)-2-chlorophenyl 2,5-dimethyl-3-thienyl ketone (compound 117)
3-methyl-2-furyl 4-(2-tolylamino)-2-chlorophenyl ketone (compound 118)
5-methyl-2-furyl 4-(2-tolylamino)-2-chlorophenyl ketone (compound 121)
4-(2-tolylamino)-2-chlorophenyl 2-pyridyl ketone (compound 122)
4-(2-tolylamino)-2-chlorophenyl 3-chloro-2-pyridyl ketone (compound 123)
1-methyl-2-imidazolyl 4-(2-tolylamino)-2-chlorophenyl ketone (compound 126)
1-methyl-2-imidazolyl 4-(2-nitrophenyl)amino-2-chlorophenyl ketone (compound 127)
1-methyl-2-imidazolyl 4-(2-aminophenylamino)-2-chlorophenyl ketone (compound 128)
3,5-dimethyl-4-isoxazolyl 4-(2-tolylamino)-2-chlorophenyl ketone (compound 129)
4-(2-tolylamino)-2-chlorophenyl 4,5-dimethyl-2-thiazolyl ketone (compound 130)
4-(2-tolylamino)-2-chlorophenyl 5-methyl-2-thienyl ketone (compound 131)
4-(2-benzonitrileamino)-2-chlorophenyl 3-chloro-2-thienyl ketone (compound 132)
4-(2-trifluoromethylphenylamino)-2-chlorophenyl 3-chloro-2-thienyl ketone (compound 133)
4-(4-bromo-2-tolylamino)-2-chlorophenyl 3-chloro-2-thienyl ketone (compound 134)
4-(4-bromo-2-cyanophenylamino)-2-chlorophenyl 3-chloro-2-thienyl ketone (compound 135)
4-(4-isoquinolylamino)-2-chlorophenyl 3-chloro-2-thienyl ketone (compound 136)
4-(4-bromo-2,3,5,6-tetramethylphenylamino)-2-chlorophenyl 3-chloro-2-thienyl ketone (compound 137)
4-(4-bromo-2-chlorophenylamino)-2-chlorophenyl 3-chloro-2-thienyl ketone (compound 138)
4-(4-bromo-2-nitrophenylamino)-2-chlorophenyl 3-chloro-2-thienyl ketone (compound 139)
4-(2-amino-4-bromophenylamino)-2-chlorophenyl 3-chloro-2-thienyl ketone (compound 140)
4-phenylamino-2-chlorophenyl 3-chloro-2-thienyl ketone (compound 141)
4-(4-bromo-2-trifluoromethylphenylamino)-2-chlorophenyl 3-chloro-2-thienyl ketone (compound 142)
4-(3-pyridylamino)-2-chlorophenyl 3-chloro-2-thienyl ketone (compound 143)
1-[4-(2-tolylamino)-2-chlorobenzoyl]pyrrole (Compound 146)
4-(2-tolylamino)-2-chlorophenyl 3,5-dimethyl-2-pyrrolyl ketone (compound 149)
4-(2-amino-4-trifluoromethylphenylamino)-2-chlorophenyl 3-chloro-2-thienyl ketone (compound 150)
4-(2-tolylamino)-2-chlorophenyl 3-chloro-4-pyridyl ketone (compound 151)
5-chloro-3-methyl-2-benzothienyl 4-(2-tolylamino)-2-chlorophenyl ketone (compound 152)
2-benzofuranyl 4-(2-tolylamino)-2-chlorophenyl ketone (Compound 153)
3-benzothienyl 4-(2-tolylamino)-2-chlorophenyl ketone (Compound 154)
3,5-dimethyl-2-benzothienyl 4-(2-tolylamino )-2-chlorophenyl ketone (Compound 155)
4-(2-tolylamino)-2-chlorophenyl 3-chloro-2-pyrazinyl ketone (Compound 156)
1-[4-(2-tolylamino)-2-chlorobenzoyl]indole (Compound 159)
4-(2-tolylamino)-2-chlorophenyl 2,5-dichloro-3-thienyl ketone (Compound 160)
5-indolyl 4-(2-tolylamino)-2-chlorophenyl ketone (Compound 161)
4-(2-tolylamino)-2-chlorophenyl 3-chloro-1-oxidopyridin-2-yl ketone (Compound 162)
4-[methyl(2-tolyl)amino]-2-chlorophenyl 3-chloro-2-thienyl ketone (Compound 163)
4-(2-tolylamino)-2-chlorophenyl 3-chloro-2-thienyl thioketone
4-(2-nitrophenylamino)-2-chlorophenyl 3-chloro-2-thienyl thioketone
4-(2-aminophenylamino)-2-chlorophenyl 3-chloro-2-thienyl thioketone
4-(2-tolylamino)-2-chlorophenyl 3-methyl-2-thienyl thioketone
4-(2-nitrophenylamino)-2-chlorophenyl 3-methyl-2-thienyl thioketone
4-(2-aminophenylamino)-2-chlorophenyl 3-methyl-2-thienyl thioketone
4-(2-nitrophenylamino)-2-chlorophenyl 1-methyl-2-pyrrolyl thioketone
4-(2-aminophenylamino)-2-chlorophenyl 1-methyl-2-pyrrolyl thioketone
4-(2-tolylamino)-2-chlorophenyl 1-methyl-2-pyrrolyl thioketone
4-(2-tolylamino)-2-chlorophenyl 1,3,5-trimethyl-4-pyrazolyl thioketone
3-methyl-2-benzothienyl 4-(2-tolylamino)-2-chlorophenyl thioketone
4-(2-tolylamino)-2-chlorophenyl 4-methyl-3-thienyl thioketone
4-(2-tolylamino)-2-chlorophenyl 2,5-dimethyl-3-thienyl thioketone
3-methyl-2-furyl 4-(2-tolylamino)-2-chlorophenyl thioketone
5-methyl-2-furyl 4-(2-tolylamino)-2-chlorophenyl thioketone
4-(2-tolylamino)-2-chlorophenyl 2-pyridyl thioketone
4-(2-tolylamino)-2-chlorophenyl 3-chloro-2-pyridyl thioketone
1-methyl-2-imidazolyl 4-(2-tolylamino)-2-chlorophenyl thioketone
1-methyl-2-imidazolyl 4-(2-nitrophenyl)amino-2-chlorophenyl thioketone
1-methyl-2-imidazolyl 4-(2-aminophenylamino)-2-chlorophenyl thioketone
3,5-dimethyl-4-isoxazolyl 4-(2-tolylamino)-2-chlorophenyl thioketone
4-(2-tolylamino)-2-chlorophenyl 4,5-dimethyl-2-thiazolyl thioketone
4-(2-tolylamino)-2-chlorophenyl 5-methyl-2-thienyl thioketone
4-(2-benzonitrileamino)-2-chlorophenyl 3-chloro-2-thienyl thioketone
4-(2-trifluoromethylphenylamino)-2-chlorophenyl 3-chloro-2-thienyl thioketone
4-(4-bromo-2-tolylamino)-2-chlorophenyl 3-chloro-2-thienyl thioketone
4-(4-bromo-2-cyanophenylamino)-2-chlorophenyl 3-chloro -2-thienyl thioketone
4-(4-isoquinolylamino)-2-chlorophenyl 3-chloro-2-thienyl thioketone
4-(4-bromo-2,3,5,6-tetramethylphenylamino)-2-chlorophenyl 3-chloro-2-thienyl thioketone
4-(4-bromo-2-chlorophenylamino)-2-chlorophenyl 3-chloro-2-thienyl thioketone
4-(4-bromo-2-nitrophenylamino)-2-chlorophenyl 3-chloro-2-thienyl thioketone
4-(2-amino-4-bromophenylamino)-2-chlorophenyl 3-chloro-2-thienyl thioketone
4-phenylamino-2-chlorophenyl 3-chloro-2-thienyl thioketone
4-(4-bromo-2-trifluoromethylphenylamino)-2-chlorophenyl 3-chloro-2-thienyl thioketone
4-(3-pyridylamino)-2-chlorophenyl 3-chloro-2-thienyl thioketone
1-[4-(2-tolylamino)-2-chlorothiobenzoyl]pyrrole
4-(2-tolylamino)-2-chlorophenyl 3,5-dimethyl-2-pyrrolyl thioketone
4-(2-amino-4-trifluoromethylphenylamino)-2-chlorophenyl 3-chloro-2-thienyl thioketone
4-(2-tolylamino)-2-chlorophenyl 3-chloro-4-pyridyl thioketone
5-chloro-3-methyl-2-benzothienyl 4-(2-tolylamino)-2-chlorophenyl thioketone
2-benzofuranyl 4-(2-tolylamino)-2-chlorophenyl thioketone
3-benzothienyl 4-(2-tolylamino)-2-chlorophenyl thioketone
3,5-dimethyl-2-benzothienyl 4-(2-tolylamino)-2-chlorophenyl thioketone
4-(2-tolylamino)-2-chlorophenyl 3-chloro-2-pyrazinyl thioketone
1-[4-(2-tolylamino)-2-chlorothiobenzoyl]indole
4-(2-tolylamino)-2-chlorophenyl 2,5-dichloro-3-thienyl thioketone
5-indolyl 4-(2-tolylamino)-2-chlorophenyl thioketone
4-(2-tolylamino)-2-chlorophenyl 3-chloro-1-oxidopyridin-2-yl thioketone
4-[methyl(2-tolyl)amino]-2-chlorophenyl 3-chloro-2-thienyl thioketone
4-(2-tolylamino)-2-chlorophenyl 3-chloro-2-thienyl ketoxime
4-(2-nitrophenylamino)-2-chlorophenyl 3-chloro-2-thienyl ketoxime
4-(2-aminophenylamino)-2-chlorophenyl 3-chloro-2-thienyl ketoxime
4-(2-tolylamino)-2-chlorophenyl 3-methyl-2-thienyl ketoxime
4-(2-nitrophenylamino)-2-chlorophenyl 3-methyl-2-thienyl ketoxime
4-(2-aminophenylamino)-2-chlorophenyl 3-methyl-2-thienyl ketoxime
4-(2-nitrophenylamino)-2-chlorophenyl 1-methyl-2-pyrrolyl ketoxime
4-(2-aminophenylamino)-2-chlorophenyl 1-methyl-2-pyrrolyl ketoxime
4-(2-tolylamino)-2-chlorophenyl 1-methyl-2-pyrrolyl ketoxime
4-(2-tolylamino)-2-chlorophenyl 1,3,5-trimethyl-4-pyrazolyl ketoxime
3-methyl-2-benzothienyl 4-(2-tolylamino)-2-chlorophenyl ketoxime
4-(2-tolylamino)-2-chlorophenyl 4-methyl-3-thienyl ketoxime
4-(2-tolylamino)-2-chlorophenyl 2,5-dimethyl-3-thienyl ketoxime
3-methyl-2-furyl 4-(2-tolylamino)-2-chlorophenyl ketoxime
5-methyl-2-furyl 4-(2-tolylamino)-2-chlorophenyl ketoxime
4-(2-tolylamino)-2-chlorophenyl 2-pyridyl ketoxime
4-(2-tolylamino)-2-chlorophenyl 3-chloro-2-pyridyl ketoxime
1-methyl-2-imidazolyl 4-(2-tolylamino)-2-chlorophenyl ketoxime
1-methyl-2-imidazolyl 4-(2-nitrophenyl)amino-2-chlorophenyl ketoxime
1-methyl-2-imidazolyl 4-(2-aminophenylamino)-2-chlorophenyl ketoxime
3,5-dimethyl-4-isoxazolyl 4-(2-tolylamino)-2-chlorophenyl ketoxime
4-(2-tolylamino)-2-chlorophenyl 4,5-dimethyl-2-thiazolyl ketoxime
4-(2-tolylamino)-2-chlorophenyl 5-methyl-2-thienyl ketoxime
4-(2-benzonitrileamino)-2-chlorophenyl 3-chloro-2-thienyl ketoxime
4-(2-trifluoromethylphenylamino)-2-chlorophenyl 3-chloro-2-thienyl ketoxime
4-(4-bromo-2-tolylamino)-2-chlorophenyl 3-chloro-2-thienyl ketoxime
4-(4-bromo-2-cyanophenylamino)-2-chlorophenyl 3-chloro-2-thienyl ketoxime
4-(4-isoquinolylamino)-2-chlorophenyl 3-chloro-2-thienyl ketoxime
4-(4-bromo-2,3,5,6-tetramethylphenylamino)-2-chlorophenyl 3-chloro-2-thienyl ketoxime
4-(4-bromo-2-chlorophenylamino)-2-chlorophenyl 3-chloro-2-thienyl ketoxime
4-(4-bromo-2-nitrophenylamino)-2-chlorophenyl 3-chloro-2-thienyl ketoxime
4-(2-amino-4-bromophenylamino)-2-chlorophenyl 3-chloro-2-thienyl ketoxime
4-phenylamino-2-chlorophenyl 3-chloro-2-thienyl ketoxime
4-(4-bromo-2-trifluoromethylphenylamino)-2-chlorophenyl 3-chloro-2-thienyl ketoxime
4-(3-pyridylamino)-2-chlorophenyl 3-chloro-2-thienyl ketoxime
1-[4-(2-tolylamino)-2-chlorobenzoxime]pyrrole
4-(2-tolylamino)-2-chlorophenyl 3,5-dimethyl-2-pyrrolyl ketoxime
4-(2-amino-4-trifluoromethylphenylamino)-2-chlorophenyl 3-chloro-2-thienyl ketoxime
4-(2-tolylamino)-2-chlorophenyl 3-chloro-4-pyridyl ketoxime
5-chloro-3-methyl-2-benzothienyl 4-(2-tolylamino)-2-chlorophenyl ketoxime
2-benzofuranyl 4-(2-tolylamino)-2-chlorophenyl ketoxime
3-benzothienyl 4-(2-tolylamino)-2-chlorophenyl ketoxime
3,5-dimethyl-2-benzothienyl 4-(2-tolylamino )-2-chlorophenyl ketoxime
4-(2-tolylamino)-2-chlorophenyl 3-chloro-2-pyrazinyl ketoxime
1-[4-(2-tolylamino)-2-chlorobenzoxime]indole
4-(2-tolylamino)-2-chlorophenyl 2,5-dichloro-3-thienyl ketoxime
5-indolyl 4-(2-tolylamino)-2-chlorophenyl ketoxime
4-(2-tolylamino)-2-chlorophenyl 3-chloro-1-oxidopyridin-2-yl ketoxime
4-[methyl(2-tolyl)amino]-2-chlorophenyl 3-chloro-2-thienyl ketoxime.

21. A pharmaceutical composition comprising, as an active component, a compound of general formula I according to any of claims 1-20 optionally together with a pharmaceutically acceptable excipient or carrier.

22. A composition according to claim 21, wherein the amount of active component is in the range of from about 0.1 to about 100% by weight of the composition.

23. A composition according to claims 21 or 22 which is in unit dosage form comprising the active component in an amount in the range of from about 0.05 to about 1000 mg.

24. A composition according to any of claims 21-23 comprising one or more other active components selected from the group consisting of glucocorticoids, vitamin D and vitamin D analogues, antihistamines, platelet activating factor (PAF) antagonists, anticholinergic agents, methylxanthines, β-adrenergic agents, COX-2 inhibitors, salicylates, indomethacin, flufenamate, naproxen, timegadine, gold salts, penicillamine, serum cholesterol lowering agents, retinoids, zinc salts and salicylazosulfapyridine.

25. A compound of the general formula I according to any of claims 1-20 for use in therapy.

26. Use of a compound of general formula I according to any of claims 1-20 for preparing a medicament for the prevention or treatment of inflammatory diseases or conditions.

27. The use of claim 26 wherein the inflammatory disease or condition is selected from the group consisting of asthma, arthritis, including rheumatoid arthritis and spondyloarthritis, gout, atherosclerosis, inflammatory bowel disease, Crohn's disease, proliferative and inflammatory skin disorders, such as psoriasis, atopic dermatitis and acne vulgaris, uveitis, sepsis, septic shock and osteoporosis.

28. The use of claims 26 or 27 additionally comprising administering one or more other active components selected from the group consisting of glucocorticoids, vitamin D and vitamin D analogues, antihistamines, platelet activating factor (PAF) antagonists, anticholinergic agents, methylxanthines, β-adrenergic agents, COX-2 inhibitors, salicylates, infomethacin, flufenamate, naproxen, timegadine, gold salts, penicillamine, serum cholesterol lowering agents, retinoids, zinc salts and salicylazosulfapyridine.

29. A method of preventing or treating inflammatory diseases or conditions, the method comprising administering, to a patient in need thereof, an effective amount of a compound of general formula I according to any of claims 1-20.

30. A method according to claim 29 wherein a compound according to any of claims 1-20 is administered sequentially or concomitantly with a compound seleceted from the group consisting of glucocorticoids, vitamin D and vitamin D analogues, antihistamines, platelet activating factor (PAF) antagonists, anticholinergic agents, methylxanthines, β-adrenergic agents, COX-2 inhibitors, salicylates, infomethacin, flufenamate, naproxen, timegadine, gold salts, penicillamine, serum cholesterol lowering agents, retinoids, zinc salts and salicylazosulfapyridine.
